(19) United States
(12) Patent Application Publication
(10) Pub. No.: US 2021/0030607 A1
(43) Pub. Date: Feb. 4, 2021

(54) DEVICES AND SYSTEMS FOR TRANSPORTING INFANTS

(71) Applicant: Tortle Products LLC, Greenwood Village, CO (US)

(72) Inventors: Jane SCOTT, Castle Rock, CO (US); Cameron FITCH, Franktown, CO (US)

(21) Appl. No.: 16/640,799

(22) PCT Filed: Aug. 21, 2018
(86) PCT No.: PCT/US2018/047301

§ 371 Date: Feb. 21, 2020

Related U.S. Application Data
(60) Provisional application No. 62/572,801, filed on Oct. 16, 2017.
(60) Provisional application No. 62/548,257, filed on Aug. 21, 2017.

(51) Int. Cl. A61G 1/044; A61G 11/00; A47C 27/08; A41D 13/05; G10K 11/162

(52) U.S. Cl. CPC A61G 1/044; A61G 11/00; A47C 27/088; A61G 2203/46; A41D 13/0568; G10K 11/162; A61G 2200/14; A41D 13/0562

(57) ABSTRACT

The present invention provides devices and system useful for transporting infants, minimizing vibrations, noise, and associated with transport. Devices of the invention include a head gear assembly, one or more body support rolls for elevating portions of the infants body, and a mattress system with a removable insert. The invention further provides methods of use associated with such devices, including methods of stabilizing a position of an infant, reducing shock or vibration to an infant during transportation, reducing shock, vibration, or noise exposure to an infant during transportation, reducing shock or vibration while providing elevation to specific portions of an infant during transportation, and reducing the risk of intraventricular hemorrhage and/or persistent pulmonary hypertension to an infant during transportation.

### BACKGROUND

Infants are especially sensitive to forces and noises present during transportation. For example, in cases involving medical transport, infants (especially micro-preemie infants) have an increased chance of suffering long-term damage, such as damage associated with intraventricular hemorrhage and/or persistent pulmonary hypertension.

Thus, there is a need in the field for devices and systems to reduce shock, vibrations, and noise experienced by infants (e.g., infants weighing about 10 pounds or less) during relocation and transportation.

### SUMMARY OF THE INVENTION

Devices for the secure transport of an infant are disclosed. The devices, including a head gear assembly, a mattress, a support roll, and a mattress system, enable secure transport of an infant such that the risk of injuring the infant during motion and/or noise exposure is reduced. The invention also includes methods of using the devices, e.g., to reduce the risk of intraventricular hemorrhage (IVH).

The present invention provides a head gear assembly configured to immobilize a head of an infant and reduce noise exposure to the infant during transport, the head gear assembly having two ear protectors, each including a housing having an inside recess and a perimeter, wherein the inside recess of the housing includes a sound-absorbing material, wherein the perimeter of the housing includes a cushioning material to contact the infant's head; a support structure having two stabilizing elements lateral to the ear protectors, the support structure configured to resist rotation of the head; and a securing element configured to exert a pressure (e.g., a slight compressive pressure, e.g., compressive force) against the head to secure the ear protectors and the support structure to the head. The securing element may directly exert the pressure (e.g., a slight compressive pressure, e.g., compressive force), e.g., by directly contacting the head, or the pressure (e.g., a slight compressive pressure, e.g., compressive force) may be indirect, e.g., transmitted through a material (e.g., a securing element). In some embodiments, all or a portion of each ear protector is between the head and one of the stabilizing elements when the head gear assembly is installed. In one embodiment, then two ear protectors, the support structure, and the securing element are integrally formed. In some embodiments, the securing elements is an elastic fabric, e.g., an elastic fabric having a hook-and-loop attachment mechanism.

Any suitable impact-resistant material may be used as the material of the housing of the ear protector. In some embodiments, the impact resistant material is polytetrafluoroethylene, polyethylene terephthalate, polypropylene, polyether ether ketone, polyoxymethylene, fluorinated ethylene propylene, perfluoroalkoxy, ethylene tetrafluoroethylene, polyvinylidene fluoride, or a copolymer thereof. In some embodiments, the sound-absorbing material on the inside recess of the housing of the ear protector is polyurethane, polyether, polyester, melamine, or a combination thereof. In some embodiments, the cushioning material of the perimeter of the ear protector is polyurethane, polyether, polyester, melamine, a liquid cushion, a gel cushion, or a combination thereof.

In further embodiments, the head gear assembly includes one or more securing elements for an external medical device, such as a CPAP mask or prongs, a nasal cannula, IV tube, or feeding tube(s). The securing element may be a strap with that includes an attachment mechanism, in particular a hook-and-loop mechanism.

The present invention further provides a support roll (or a plurality of support rolls) for stabilizing a position of an infant, the support roll having a wedge-shaped cross-section, where the support roll has a length from 6 to 24 inches, a thickness from 1 to 4 inches, and a maximum width from 3 to 6 inches. In some embodiments, the support roll is encased in a fabric, where the fabric is connected to a strap having a first end and a second end, wherein the first end is connected to the fabric at a portion of the fabric contacting the vertex of the wedge and the second end is removably attachable to the fabric. In some embodiments, the support roll is made from polyurethane and is in the shape of a crescent. In some embodiments, the width of the strap of the support roll decreases from the first end to the second end. In one embodiment, the second end of the strap includes an attachment mechanism, in particular a hook-and-loop mechanism.

The present invention also provides a mattress configured to support an infant's body having a top surface, a bottom surface, and a core material therebetween, wherein the core material has a plurality of conduits running vertically therethrough, and wherein the plurality of conduits provides a variable compressive modulus over the top surface of the mattress. In one embodiment, the mattress is a removable mattress configured to nestably engage with a peripheral mattress, where the peripheral mattress includes a cutout configured to contact the periphery of the removable mattress to resist lateral motion of the removable mattress. In some embodiments, the array of conduits enhances heat diffusivity from the bottom surface of the mattress to the top surface of the mattress, with some of the plurality of conduits having a removable member configured to fill the conduit.

Any suitable foam material may be used for the mattress. In some embodiments, the mattress is made from viscoelastic foam, polyurethane foam, memory foam, or a combination thereof. In further embodiments, the mattress is the mattress is covered in a protective material. In some embodiments, the protective material is a composite fabric, fabric blend, vinyl, acetate, or a combination thereof. In some cases, the mattress includes a plate that is disposed underneath the mattress when enclosed in a protective material. In some embodiments, the mattress is configured to bend to an incline.

The invention further provides a mattress system for secure transport of an infant, the mattress system having a peripheral mattress having a cutout; a removable mattress having a top surface, a bottom surface, and a core material therebetween having a shape configured to nestably engage within the cutout of the peripheral mattress, where upon engagement with the peripheral mattress, lateral motion of the removable mattress is reduced. In some embodiments, the peripheral mattress is sized to fit within the tray of an incubator chamber. In a further embodiment, the mattress system contains a wedge configured to be positioned below the removable mattress. In one embodiment, the wedge, when installed, provides an incline to the top surface of the removable mattress. In some embodiments, the wedge, when installed, provides an incline of between 0 and 30° to the top surface of the removable mattress (e.g., about 0° to about 30°, e.g., about 0°, about 1°, about 2°, about 3°, about 4°, about 5°, about 6°, about 7°, about 8°, about 9°, about 10°, about 15°, about 20°, about 25°, or about 30°, e.g., about 0° to about 10°, about 5° to about 15°, about 10° to about 20°, about 15° to about 25°, or about 20° to about 30°). As used herein, “about” means +/−25% of a recited value.

In certain embodiments, at least one of the mattresses of the mattress system, e.g., the peripheral mattress or the removable mattress, or both, include one or more sources of light. The one or more sources of light may be configured to provide a phototherapy, provide light for monitoring, provide a source of heat, act as status indicators for installed sensors, e.g., temperature, or a combination thereof. In some embodiments, the one or more sources of light is a light emitting diode (LED).

In further embodiments, the mattress system has a plurality of straps where each of the plurality of straps has a first end and a second end, where the first end and second end of each of the plurality of straps has a connector configured to secure the infant to the mattress. In further embodiments, the removable mattress has openings through the top surface, bottom surface, and core material for the plurality of straps to pass through. In some embodiments, at least one or more of the plurality of straps is configured to secure the head of the infant to the mattress system. In some embodiments, at least one or more of the plurality of straps is configured to secure the torso of the infant to the mattress system. In some embodiments, at least one or more of the plurality of straps is configured to secure the pelvis of the infant to the mattress system. In some embodiments, the mattress system contains the head gear assembly described herein. In some embodiments, the mattress system contains one or more support rolls as described herein.

Another aspect of the invention provides a method of stabilizing a position of an infant, the method including contacting the infant with one or more support rolls to elevate a first portion of the infant's body relative to a second portion of the infant's body, wherein the first portion of the infant's body is lateral to the infant's midline (e.g., an arm or leg), superior to the infant's waist (e.g., head or shoulder), or inferior to the infant's waist (e.g., a leg). In one embodiment, the support roll has a wedge-shaped cross section. In some embodiments, support rolls elevate a first portion of the infant's body between an angle from about 0° to about 45° (e.g., about 0°, about 1°, about 2°, about 3°, about 4°, about 5°, about 6°, about 7°, about 8°, about 9°, about 10°, about 15°, about 20°, about 25°, about 30°, about 35°, about 40°, or about 45°, e.g., about 0° to about 10°, about 5° to about 15°, about 10° to about 20°, about 15° to about 25°, about 20° to about 30°, about 25° to about 35°, about 30° to about 40°, or about 35° to about) 45°. In further embodiments, the method includes contacting the infant with one or more support rolls to elevate a third portion of the infant's body relative to the second portion of the infant's body. In some embodiments, the elevation reduces the risk of Trendelenburg (e.g., during transport). In some embodiments, the elevation reduces the risk of heightened intracranial pressure.

In another aspect, the invention provides a method of reducing shock or vibration to an infant during transportation by positioning the infant on any of the mattress systems described herein. In one embodiment, the infant is secured to the mattress system using a plurality of straps. In some embodiments, the mattress system is housed within an incubator chamber. In some embodiments, a portion of the infant's body is elevated by a plurality of support rolls. In some embodiments, the ears of the infant are covered by a head gear assembly configured to reduce noise exposure. For example, the head gear assembly may be any head gear assemblies described herein.

In another aspect, the invention provides a method of reducing shock, vibration, or noise exposure to an infant during transportation the method including providing a mattress system, providing a head gear assembly, and securing the head gear assembly to the infant and securing the infant to the mattress system. In some embodiments, the mattress system is housed within an incubator chamber. In some embodiments, a portion of the infant's body is elevated by a plurality of support rolls, e.g., a plurality of any of the support rolls described herein.

In another aspect, the invention provides a method of reducing shock or vibration while providing elevation to specific portions of an infant during transportation, the method including providing a mattress system, providing a plurality of support rolls, and securing the head gear assembly to the infant and securing the infant to the mattress system. In some embodiments, the mattress system is housed within an incubator chamber. In some embodiments, the ears of the infant are covered by a head gear assembly configured to reduce noise exposure. For example, the head gear assembly may be any head gear assemblies described herein.

In a further aspect, the invention provides a method of reducing the risk of IVH and/or persistent pulmonary hypertension (PPHN) to an infant during transportation using any of the methods described herein. In one embodiment, the method includes providing a mattress system described herein, providing a head gear assembly described herein, providing a plurality of support rolls described herein, and securing the infant to the mattress system using the plurality of straps of the mattress system.

### BRIEF DESCRIPTION OF THE DRAWINGS

FIG. 1 is a photograph showing a plastic outer shell of an ear protector. Slots configured to accept one or more straps are also shown.

FIG. 2 is a photograph showing the inside of an ear protector. The inside of the ear protector contains a sound dampening foam, which is surrounded at the perimeter by a separate foam. The perimeter foam is configured to cup the infant's ear and to protect the skin of the infant.

FIG. 3 is a photograph showing a side view of an ear protector.

FIG. 4 is a photograph showing a doll wearing head gear assembly including ear protectors on both ears (i.e., bilaterally).

FIG. 5 is a photograph showing a doll in partial side lying using a system of the invention. The doll is wearing head gear assembly having a single ear protector.

FIG. 6 is a photograph of exemplary head gear assemblies to be worn by the infants during transport. The mattresses are shown for scale.

FIG. 7 is a photograph of support structures for use as part of a head gear assembly of the invention. The support structures have air spaces, which have been made by drilling. The support structures are configured to dampen vibrations and sound during transport.

FIG. 8 is a rear photograph of an infant on a mattress system of the invention where the support structures of a head gear assembly are positioned lateral to the head of the infant. The support structures are also secured to the mattress system by the straps crossed at the bottom of the picture.

FIG. 9 is a side photograph of an infant on a mattress system of the invention showing the support structures of a head gear assembly secured below the mattress by a strap.

FIG. 10 is a photograph of a securing element of the head gear assembly originating with a wedge plate of the mattress system of the invention.

FIG. 11 is a photograph of a support foam roll and securing element used to secure an infant in position. The support foam roll is made of soft memory foam and helps to position the infant in a “fetal flexed position” while securing the infant for transport. The support form roll also functions to dampen vibrations.

FIG. 12 is a photograph showing a memory foam piece for use as part of a support roll. The hand provides a size comparison.

FIG. 13 is a photograph showing a support roll having a FABRIFOAM® cover and a long strap to secure the infant in position. The strap includes a hook-and-loop (VELCRO®) piece to secure to a corresponding piece, or to the FABRIFOAM®, as needed.

FIG. 14 is a photograph showing a doll lying on a full mattress suitable for a transport isolation tray. Also shown is the head gear assembly and support roll including a strap holding the doll in a partial lateral position.

FIG. 15 is a photograph showing a micro-preemie doll with head gear assembly and support roll lying on a removable mattress in a supine midline position. One or two support rolls may be used to secure the infant on either side.

FIG. 16 is a photograph of a pair of body support rolls of the invention that are connected together by a foot support roll of the invention.

FIG. 17 is a side view photograph showing a doll wearing a head gear assembly laying on a removable mattress mated with the cutout of a peripheral mattress inside of a transport isolation system. The black object adjacent to the doll is a support roll to provide support for the doll during transport. The 15-30° elevation of the head portion of the removable mattress is visible.

FIG. 18 is a front view photograph showing a doll wearing a head gear assembly laying on a removable mattress mated with the cutout of a peripheral mattress inside of a transport isolation system. The black object located below the doll is a support roll to provide support for the doll during transport. The feet of the doll are supported by a curved support roll. The 15-30° elevation of the head portion of the removable mattress is visible.

FIG. 19 is an end on view photograph showing a doll wearing a head gear assembly laying on a removable mattress mated with the cutout of a rectangular mattress inside of a transport isolation system. The black objects located on both sides below the doll are support rolls to provide support for the doll during transport. The feet of the doll are supported by a curved support roll.

FIG. 20 is a photograph showing a transport isolation tray containing a rectangular mattress.

FIG. 21 is a top view photograph of the straps used to secure the infant to the mattress system. Shown are straps for the pelvis and torso passing through the removable mattress and the straps used to secure the removable mattress to the transport isolation system.

FIG. 22 is a side view photograph of the straps used to secure the infant to the mattress system. Shown are straps for the pelvis and torso passing through the removable mattress and the straps used to secure the removable mattress to the transport isolation system. The 15-30° elevation of the head portion of the removable mattress is visible.

FIG. 23 is a top view photograph of the straps used to secure the infant to the mattress system. Shown are straps for the pelvis and torso passing through the removable mattress and the straps used to secure the removable mattress to the transport isolation system. The 15-30° elevation of the head portion of the removable mattress is visible.

FIG. 24 is a photograph of the straps that are configured to pass through cutouts in a removable mattress for securing it to the transport isolation system.

FIG. 25 is a photograph showing the straps passing through cutouts in a removable mattress and how those straps are secured to the plastic lip of the trap of a transport isolation system.

FIG. 26 is a photograph of a complete mattress system of the invention (peripheral mattress, removable mattress, wedge, head gear assembly, and the various securing straps.

FIG. 27 is a photograph of a doll secured in a complete mattress system of the invention (peripheral mattress, removable mattress, wedge, head gear assembly, and the various securing straps.

FIG. 28 is a photograph showing a doll wearing head gear assembly lying on a removable mattress mated with the cutout of a peripheral mattress fully secured (e.g., head, torso, and pelvis) to the inside of a transport isolation system. The 15-30° elevation of the head portion of the removable mattress is visible.

FIG. 29 is a photograph showing two types of mattresses. The rectangular mattress at the top is configured to fit within a transport isolation tray. A removable mattress is shown on the lower portion of the photograph, which is useful as extra support during transport. Removable mattresses are shaped to allow an operator to lift the head portion of the mattress, e.g., 15-30°, in accordance with medical protocols for treatment of premature infants (e.g., micro-preemies). A covered, smaller mattress is shown on the bottom left; a mattress foam featuring conduits in a diamond pattern in shown in the lower center; and an extra, perforated mattress foam is shown in the lower right portion of the photograph.

FIG. 30 is a photograph showing two layers of a mattress of the invention.

FIG. 31 is a close-up photograph showing a removable mattress foam featuring conduits formed in a diamond pattern.

FIG. 32 is a close-up photograph showing a removable mattress having wedge inserted at the head end of the removable mattress with the removable mattress and wedge covered by a protective cover.

FIG. 33 is a close-up photograph of an uncovered removable mattresses featuring an open diamond pattern of conduits in which selected conduits are filled with diamond shaped removable inserts.

FIG. 34 is a close-up photograph of an uncovered removable mattresses featuring an open diamond pattern of conduits in which selected conduits are filled with diamond shaped removable inserts FIG. 35 is a close-up photograph of a rectangular mattresses featuring cylindrical conduits in which selected conduits are filled with cylindrical shaped removable inserts.

FIG. 36 is a photograph of a covered removable mattress featuring an open diamond pattern of conduits.

FIG. 37 is a schematic of the underside of an uncovered removable mattress featuring an open pattern of cylindrical conduits and channels within the mattress to define support zones in the mattress.

FIG. 38 is a schematic of the top of an uncovered removable mattress featuring an open pattern of cylindrical conduits and a horseshoe shaped support structure for the head of an infant.

FIG. 39 is a photograph of a cover for a removable mattress.

FIG. 40 is a photograph of a disassembled removable mattress of the invention. The cover is shown at left, the mattress in the center, and the plate at right, with the mattress and plate having a substantially similar pattern of cylindrical conduits.

FIG. 41 is a side view photograph of a wedge configured to be placed below the removable mattress. The wedge is a horseshoe-shaped piece of foam covered with a plastic plate to provide support for the removable mattress with conduits to permit heat transfer. Also shown is the edge material used to keep the wedge in place when engaged with the peripheral mattress.

FIG. 42 is a top view photograph of a wedge configured to be placed below the removable mattress. The wedge is a horseshoe-shaped piece of foam covered with a plastic plate to provide support for the removable mattress with conduits to permit heat transfer. Also shown is the edge material used to keep the wedge in place when engaged with the peripheral mattress.

FIG. 43 is a top view photograph of a wedge configured to be placed below the removable mattress. The wedge is a horseshoe-shaped piece of a solid polymer and includes polymer feet on its underside to prevent motion.

FIGS. 44A-44C are photographs of removable mattresses of the invention containing one (FIGS. 44A and 44B) or more (FIG. 44C) sources of blue light. FIG. 44A shows the source of blue light illuminated inside a mattress. FIG. 44B shows the outer cover of a mattress opened to indicate the installation location of the source of blue light. FIG. 44C shows an alternative embodiment containing three parallel strips of blue light in the mattress.

FIGS. 45A-48E are photographs of an embodiment of a method of the invention for securing an infant in a mattress system of the invention.

FIGS. 49A-49C are photographs of accessories that may be utilized with a mattress system of the invention. FIG. 49A shows a CPAP mask secured to an embodiment of a head gear member of the invention using hook and loop securing elements. FIG. 49B shows a nasal cannula secured to an embodiment of a head gear member of the invention using hook and loop securing elements. FIG. 49C shows a decorative bilishade placed over the eyes of an infant and secured to an embodiment of a head gear member of the invention using hook and loop securing elements.

FIGS. 50A and 50B are photographs of an uncovered removable mattress featuring a depression on the top side of the mattress (FIG. 50A) and a support ring on the bottom side of the mattress (FIG. 50B).

FIGS. 51A and 51B are photographs of an embodiment of the mattress having lateral loops on the side of the mattress. FIG. 51A shows a configuration in which a strap passes through one or more of the loops on the side of the mattress in addition to passing through a cutout in the tray. FIG. 51B shows a configuration in which the strap passes through loops on the side of the mattress without passing through a cutout in the tray, thereby allowing for uninterrupted removal of the mattress from the tray/peripheral mattress assembly.

### DETAILED DESCRIPTION OF THE INVENTION

The present invention provides devices and systems (e.g., medical devices and systems) designed to reduce shock, vibration, and/or sound to an infant during relocation or transportation from one location to another. Devices and systems of the invention can be configured to reduce vibration and/or shock, including vibration associated with fixed wing aircraft, and helicopter take offs, landings, the use of thrust reversers, rotor blade vibration, turbulence, road noise, and road surface conditions. In some embodiments, devices and systems of the invention are configured for use with infants having a weight of less than about 10 pounds (about 4500 grams). Methods of the invention include methods of reducing the likelihood of injury, such as intraventricular hemorrhage (IVH) or persistent pulmonary hypertension (PPHN).

### Head Gear Assembly

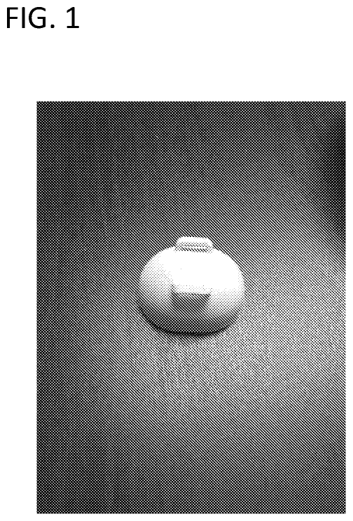

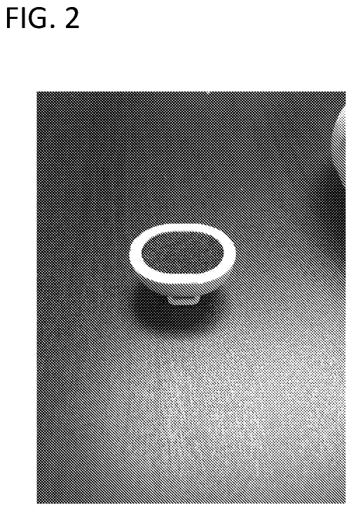

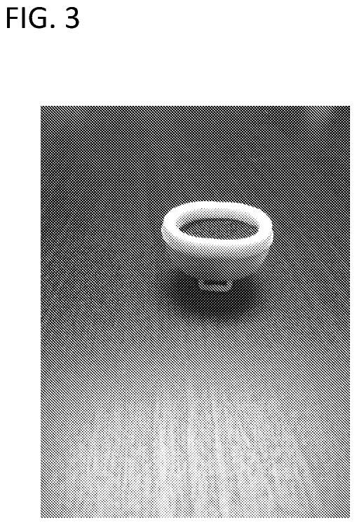

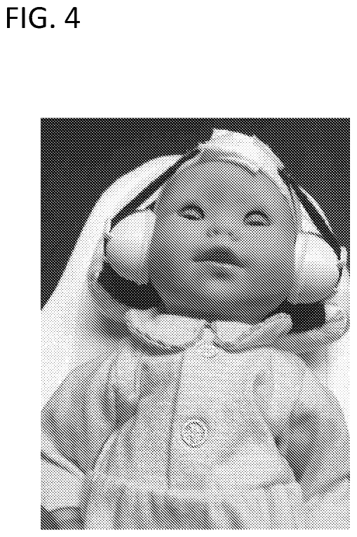

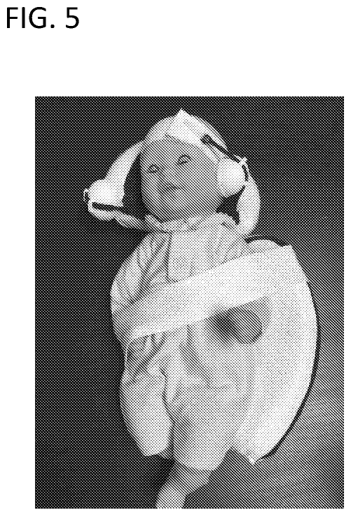

The invention includes a head gear assembly having two ear protectors which can be used together with a support structure and/or a securing element to cover the ears to dampen the sounds from any source and to protect the structures of the outer ear from impacts due to unintended motion during transport. Alternatively, the ear protectors, support structure, and securing element may be used as separate pieces, with the ear protectors and support structure secured to the infant's head with the securing element. Exemplary ear protectors are shown in FIGS. 1-3, and head gear assembly combing ear protectors, support structures, and securing elements are shown in FIGS. 4-6.

In some embodiments, the ear protectors may have a hard outer shell and a sound absorbing foam cushion that may also act to reduce trauma to the ear due to unintended impact during transport as shown in FIGS. 1-3. The outer perimeter of the ear protector may include a cushioning material that is placed against the skin when the ear protector is installed on the infant. The cushioning material surrounding the perimeter of the ear protector may be a foam, such as polyurethane, polyether, polyester, melamine, or may be a liquid-based or gel-based cushion material. The sound absorbing foam may be made from any material that can dissipate pressure waves, such as polyurethane, polyether, polyester, and melamine. Other sound absorbing foam materials are known in the art. The outer shell of the ear protector can be made of any suitable material, (e.g., a polymer) that is rigid, durable, and physiologically inert, such as polytetrafluoroethylene, polyethylene terephthalate, polypropylene, polyether ether ketone, polyoxymethylene, fluorinated ethylene propylene, perfluoroalkoxy, ethylene tetrafluoroethylene, polyvinylidene fluoride, or copolymers thereof. Other suitable materials for the outer shell of the ear protectors, such as metals, are known in the art.

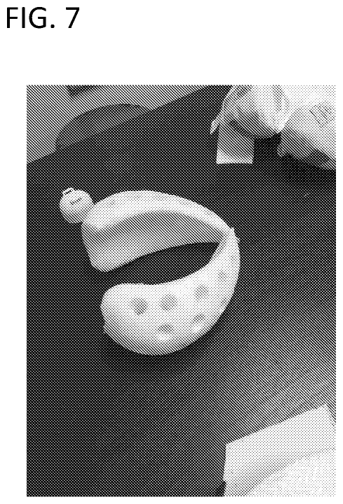

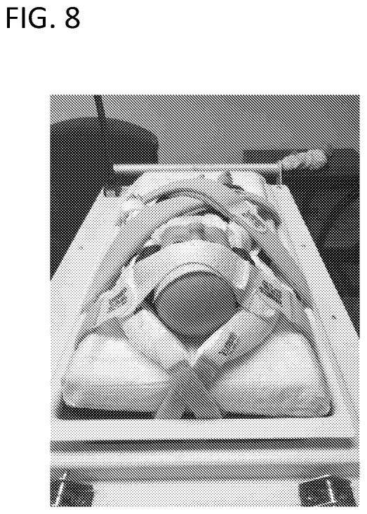

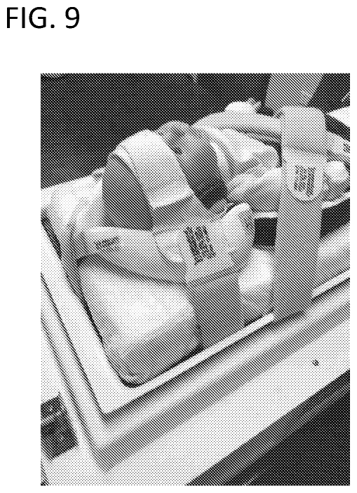

An example of a support structure designed for use with ear protectors is shown in FIG. 7. In this configuration, the support structure includes two separate supports, i.e., one support per ear protector, disposed lateral to the ear protector. The supports may be used independently of each other and may include securing element(s) to connect them to a portion of the transport system using a suitable fastener, such as hook and loop or a snap. In another embodiment, the two supports are integrated into a single head gear assembly that can be placed around the head of the infant. Alternatively, the support structure for the ear protectors may be a single unit with dimensions sufficient to span the distance between the ear protectors (e.g., having a horseshoe shape). In a particular embodiment, the support structure for use with ear protectors has dimples or holes, which may be made by, e.g., drilling. The support structures are configured to dampen vibrations and sound during transport. The area of contact of the support structure of the head gear is directly related to the weight of the infant's head, and helps to reduce the impact of shock and vibration. In addition to providing support for the ear protectors, the support structure also provides support for the head of the infant by prevent it from freely rotating from side to side during transportation. In one embodiment, when the head gear assembly is configured with two stabilizing elements as shown in FIGS. 8 and 9, the spacing between the two stabilizing elements is adjustable. This adjustable spacing allows for the head of an infant to be elevated above the plane of the mattress. For example, the stabilizing elements may be used to provide sufficient elevation to the head to keep the airway of an infant open during transport

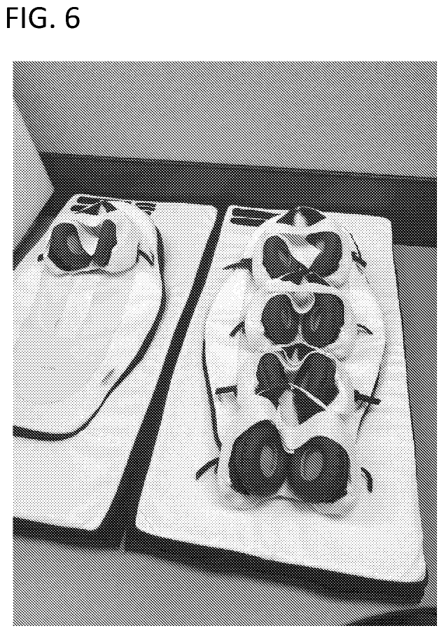

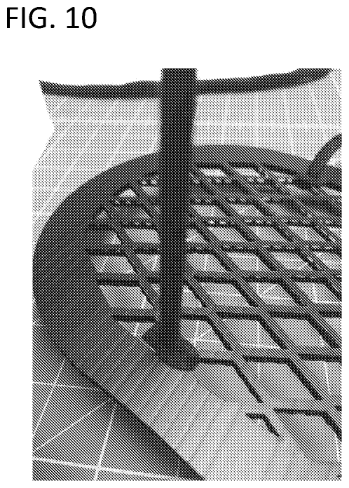

In some embodiments, the ear protectors and support structure may be held in position on the head of an infant by a securing element, e.g., a cover and/or a strap, attached directly to the ear protector (such as the example shown in FIGS. 1-5 where the ear protector has a molded slot in the outer shell for a strap to pass through). Alternatively, the ear protectors, support structure, and securing elements may be integrally formed. For example, FIG. 6 shows an exemplary head gear assembly, where the ear protectors and support structure are nested together (and fully enveloped) by an elastic fabric securing element having a strap to secure the assembly around the head of an infant. The securing element for the head gear assembly as shown in FIGS. 8 and 9, may connect to the support structure in the front, e.g., the securing element does not cross the crown of the head. In this configuration, the securing element attaches to the support structure via a strap that runs to the bottom of the mattress (e.g., at a bottom plate), thereby applying a downward force to the support structures and ear protectors. This downward force, controlled by the elasticity of the material used to create the securing element, allows the ear protectors to remain in place and prevent the head of the infant from freely rotating from side to side during transportation. In some embodiments, the securing element for the head gear assembly originates from the mattress system the infant is secured to, such as shown in FIG. 10, where the securing elements for the head gear assembly are connected to a component of the mattress system.

The securing element may be made from an elastic fabric material, such as LYCRA® or neoprene. Alternatively, the securing element may be made from another suitable fabric material, such as a non-stretchable synthetic fiber, e.g., nylon, leather, or natural fiber, e.g., cotton. Other suitable materials, both elastic and non-elastic, are known in the art. The securing element, when configured as a cover (enveloping both the ear protectors and support structures) and a strap, the strap may be integrated into the cover, e.g., an end of the strap is permanently affixed to the cover, e.g., sewn on, or the strap is formed from a contiguous piece of material where a strap is formed by a length of material decreasing in width. Alternatively, the strap may be fully positionable, having both ends of the strap having an attachment system such that the ends of the strap may be affixed to the cover in a convenient location to ensure proper positioning of the ear protectors and support structure. In this configuration, the strap can be of uniform width from one end to the other, or alternatively the strap may be wider at one end than the other. For example, in a strap that is sewn into a cover for securing the ear protectors and support structure, the end of the strap sewn on may be wider that the end which releasably attaches to the cover securing the ear protectors and support structure. In another embodiment, a strap that is positionable at both ends, e.g., having an attachment system at both ends such it can be positioned anywhere on the cover securing the ear protectors and support structure, may have ends that are wider than the central region of the strap. The securing element can be affixed using any suitable attachment system, including hook and loop, e.g., VELCRO®, snaps, buckles, e.g., side release, e.g. FASTEX®, or other attachment systems known in the art.

### Support Rolls

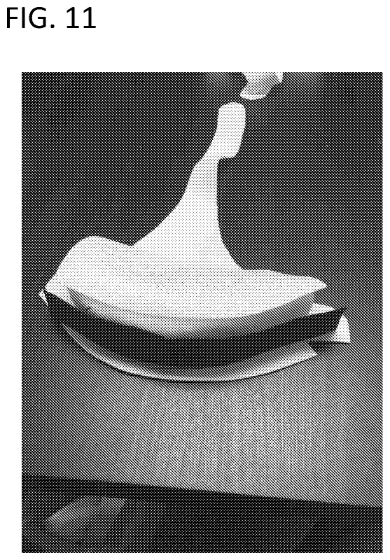

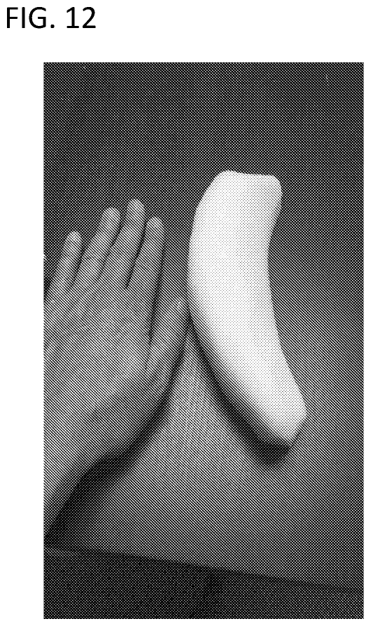

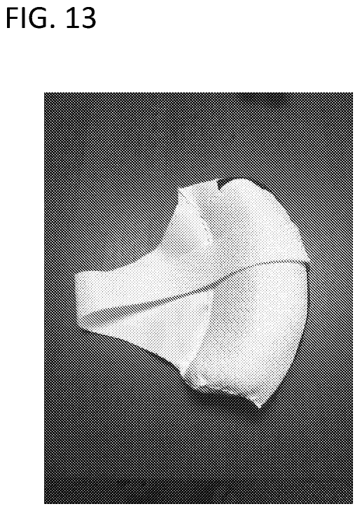

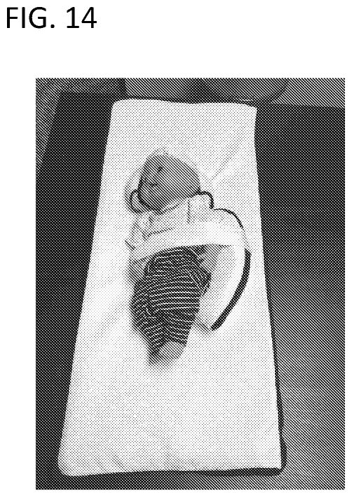

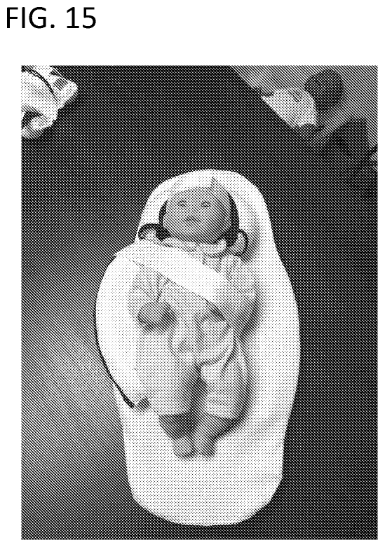

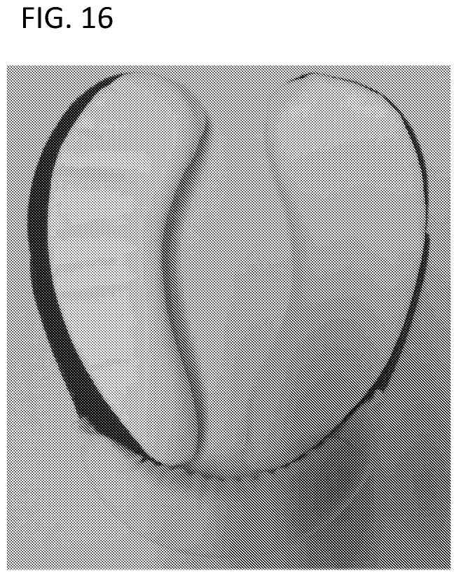

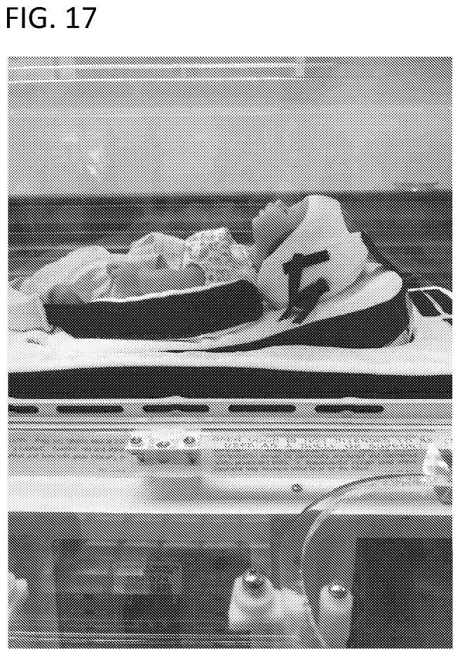

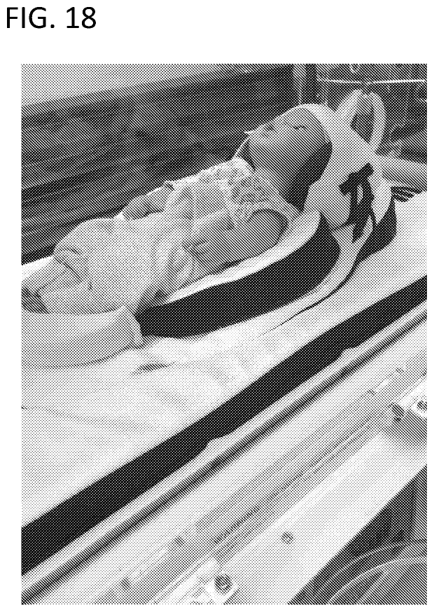

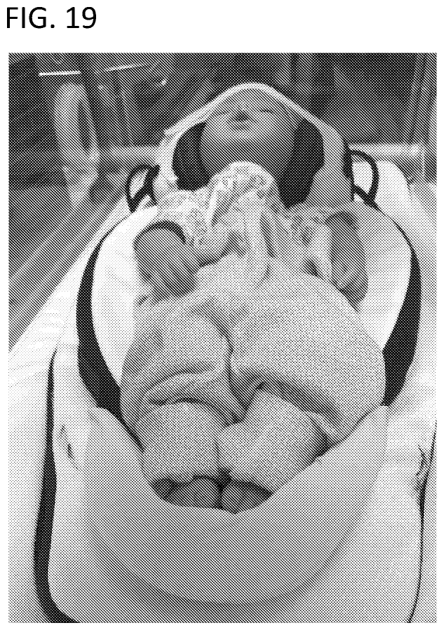

In some embodiments, the invention contains support rolls that act to provide boundaries for an infant's head and/or body, e.g., to simulate fetal conditions in utero, placing the infant in a comfortable fetal flexion. These support rolls may be placed anywhere on the infant where support and/or elevation is needed, such as beneath the torso, feet/legs, or behind the head. Multiple support rolls may be used to place the infant in the exact position required for transport. The size and shape of a support roll is dictated by which part of the body the support roll is intended to contact. In general, the support rolls may have a length from about 6 inches to about 24 inches long (e.g., about 6 inches, about 7 inches, about 8 inches, about 9 inches, about 10 inches, about 11 inches, about 12 inches, about 13 inches, about 14 inches, about 15 inches, about 16 inches, about 17 inches, about 18 inches, about 19 inches, about 20 inches, about 21 inches, about 22 inches, about 23 inches, or about 24 inches. The width of the support rolls may be from about 3 inches to about 6 inches (e.g., about 3 inches, about 3.5 inches, about 4 inches, about 4.5 inches, about 5 inches, about 5.5 inches, or about 6 inches). The thickness of the support roll will be dependent on the size of the infant where the support roll is needed as well as how much support and/or elevation is needed. In general, the support rolls may have a thickness from about 1 inch to about 4 inches (e.g., about 1 inch, about 1.5 inches, about 2 inches, about 2.5 inches, about 3 inches, about 3.5 inches, or about 4 inches). For example, FIGS. 11-13 show photographs of a single support roll designed to be placed adjacent to the torso of an infant to help position the infant in a “fetal flexed position”. The support rolls may also have a wedge shape, e.g., have a pre-defined angle that sets the position of the infant upon use of the support roll. For example, FIGS. 14 and 15 show an infant turned on its side by the use of an angled support roll. The angle can be anywhere from about 0° to about 45°, e.g., about 0°, about 1°, about 2°, about 3°, about 4°, about 5°, about 6°, about 7°, about 8°, about 9°, about 10°, about 15°, about 20°, about 25°, about 30°, about 35°, about 40°, or about 45°, e.g., about 0° to about 10°, about 5° to about 15°, about 10° to about 20°, about 15° to about 25°, about 20° to about 30°, about 25° to about 35°, about 30° to about 40°, or about 35° to about 45°. One or more support rolls may be used concurrently to provide the required support and/or elevation to one or more parts of the infant's body as medically necessary. The body support rolls may each be used as an independent support, e.g., two or more support rolls are not connected together before installation. Alternatively, two or more support rolls can be integrated into a single unit than can be placed underneath an infant. For example, FIG. 16 shows an embodiment of a body support roll consisting of two support rolls for supporting the torso on either side of the infant, each of which is connected to one side of a support roll configured to be placed at the feet of an infant. The connection may be substantially permanent, e.g., sewn together; alternatively, the connection between the support rolls may be any suitable attachment system, including hook and loop, e.g., VELCRO®, buckles, e.g. side release, e.g., FASTEX®, snaps, buttons, pins, or other suitable attachment mechanism known in the art. For example, FIGS. 17-19 show a doll secured within a mattress system of the invention where the torso is supported by two support rolls, e.g. one on each side of the torso, as well as a support roll placed at the feet of the infant.

The support rolls may be made of a suitable foam material, such as a viscoelastic foam, e.g., polyurethane, e.g., memory foam. Other suitable foams for mattresses are known in the art. The support rolls may be made of a single type of foam, e.g., polyurethane, or alternatively, each can be made of a different foam material to allow the compressibility of the support roll to be adjusted based on the amount of support required. For example, the foam the may be chosen to have a slow or fast recovery time after compression to control the level of support provided to the infant during transport. The support rolls are typically a solid piece of foam and may be covered in a protective cover. These covers are protective materials to enhance the comfort of the infant and provide additional vibration dampening. For example, the support rolls can be contained inside a cover made from a composite fabric material, such as FABRIFOAM® which may provide breathability and comfort. In some embodiments, the support rolls are not covered by a fabric cover when in use, e.g., the foam of the support roll directly contacts the body of the infant, providing a skin-like feel. In some embodiments, the support rolls are covered in a material having a skin-like feel.

Additionally, the cover of the support roll may have a securing element, e.g., a strap, to secure the support roll around the selected part of the infant's body, such as the embodiments shown in FIGS. 11-19. The strap may be integrated into the cover, e.g., an end of the strap is permanently affixed to the cover, e.g., sewn on. Alternatively, the strap may be full positionable, having both ends of the strap terminating in an attachment system such that an end of the strap may be affixed to the support roll in a convenient location. The strap can be of uniform width from one end to the other, or alternatively the strap may be wider at one end than the other. For example, in a strap that is sewn to the cover of the support roll, the end of the strap sewn on may be wider that the end which releasably attaches to the cover of the support roll. In another embodiment, a strap that is positionable at both ends, e.g., having an attachment system at both ends such it can be positioned anywhere on the support roll, may have ends that are wider than the central region of the strap. The strap can be affixed using any suitable attachment system, including hook and loop, e.g., VELCRO®, buckles, e.g. side release, e.g., FASTEX®, snaps, buttons, pins, or other suitable attachment mean known in the art.

### Mattress Systems

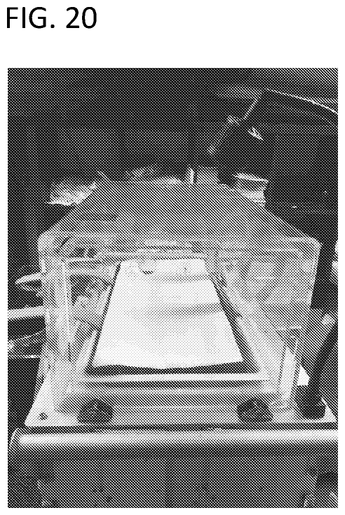

The present invention features a mattress system to be secured inside of a transport isolation tray, e.g., an ISOLETTE®, to reduce shock, vibration, and/or sound to an infant during relocation or transportation from one location to another (e.g., during emergency medical transport) such as shown in FIG. 20. In addition to providing secure transport, the mattress systems of the invention also provide an added safety benefit in the event of an accident during transport. As the mattress system is secured inside of a transport isolation system, the infant secured to the mattress is less likely to be ejected from the transport isolation system in the event of an accident during transport. When transporting an infant on a mattress, the recommended amount of compression into the mattress material for secure transport is at least 20% compression, (e.g. at least 20% compression, at least 25% compression, at least 30% compression, at least 35% compression, at least 40% compression, at least 45% compression, at least 50% compression, at least 55% compression, at least 60% compression, at least 65% compression, at least 70% compression, at least 75% compression, or at least 80% compression, e.g., 20-80%, 20-70%, 20-60%, 20-50%, 20-40%, or 20-30% compression). Insufficient compression of the body of the infant into the mattress system may lead to an unacceptably high level of exposure to vibrations, potentially causing injury during transport. The minimum acceptable compression of at least 20% may be achieved by the securing elements of the mattress system, such as the system of straps for the individual components of the system described herein.

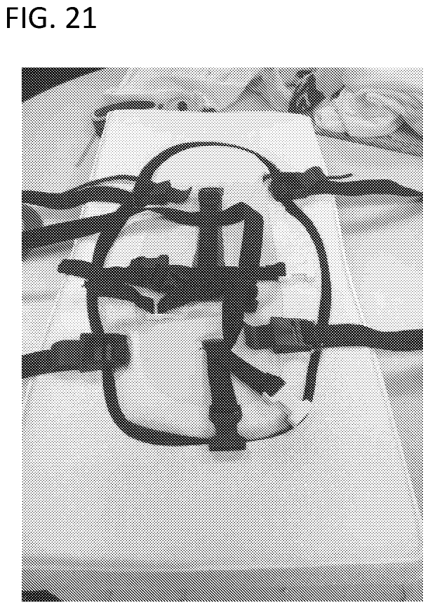

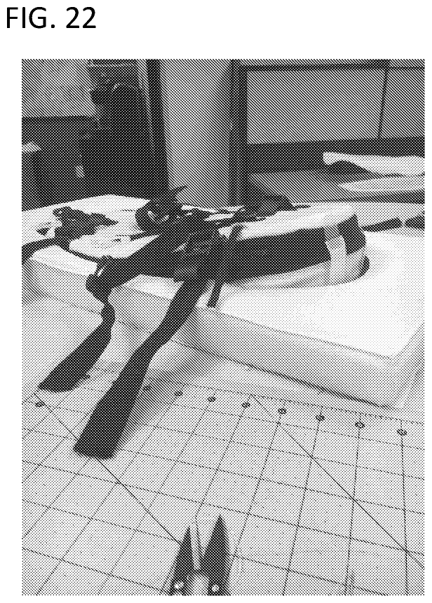

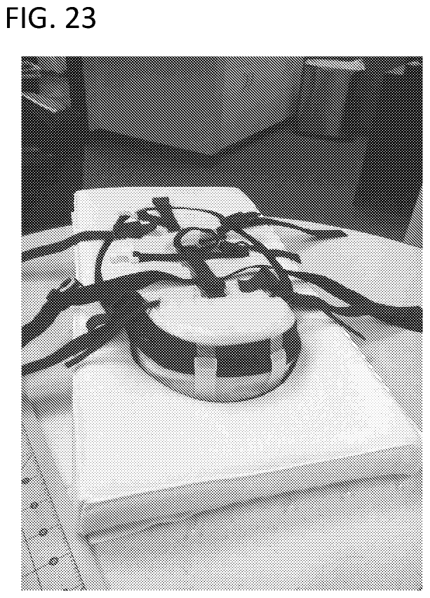

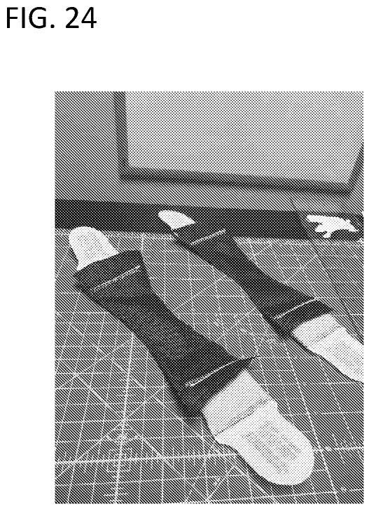

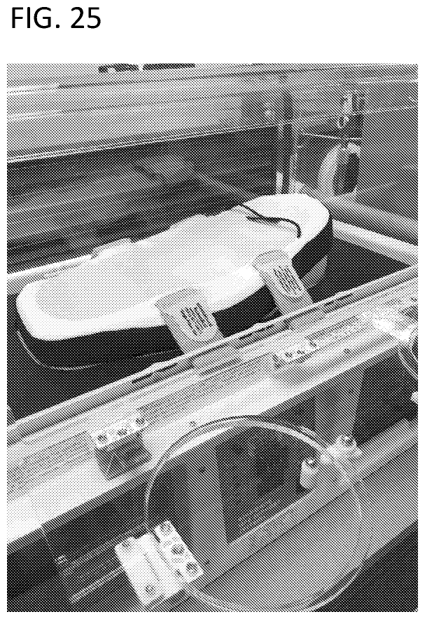

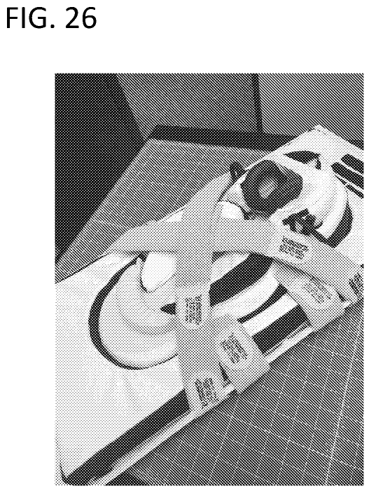

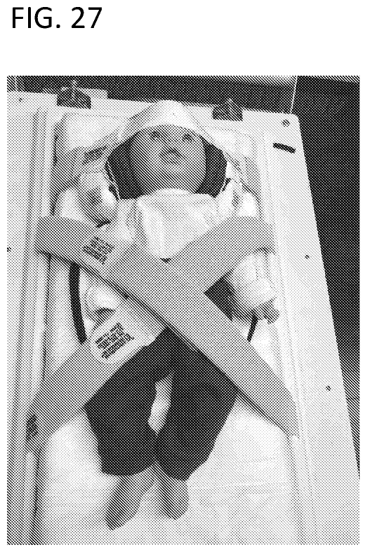

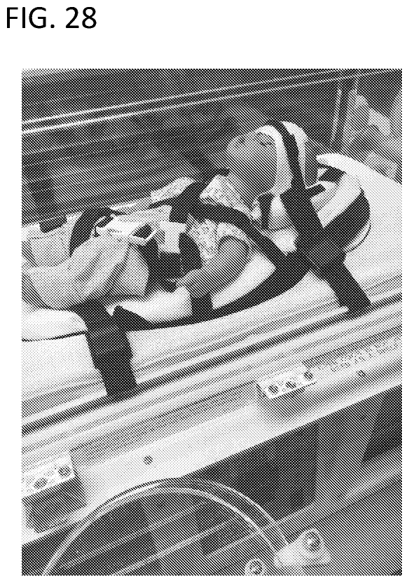

In particular, the securing elements of the components of the mattress system described herein are configured to secure the infant to a mattress system (e.g., a multi-component mattress system) at multiple points on the body, ensuring that the body of the infant is kept in proper alignment during transport. Further, mattress systems of the invention include a system of integrated straps. For example, in some instances, the straps pass through openings integrated in the mattresses (the rectangular mattress, the removable mattress, or both mattresses) of the mattress system. The integrated straps are configured to secure the infant to the mattress system, secure any support materials, e.g., support rolls, to the infant, and to secure the mattress system to the transport isolation tray. Examples of the various straps used to secure the infant to the mattress system, support rolls to the infant, and the mattress system to the transport isolation tray are shown in FIGS. 21-28. For example, FIG. 24 shows a pair of straps that may be inserted into cutouts made within the removable mattress such that the straps may be passed through the cutouts. The ends of the straps may then be attached to appropriate points on the transport isolation tray as shown in FIG. 25. Each end of the strap has a suitable connector such that the infant can be secured to mattress system and to secure the mattress system within the transport isolation tray. The locations of the straps are designed to secure the infant in a proper position and to restrict movement while in transport. The straps may be located on or near the infant's torso, legs, arms, pelvis, or head, and are sufficiently wide such that the force of compression upon tightening the straps securely is evenly distributed both over the infant and the mattress system. For example, the mattress system may contain one or more straps that may be used to secure the head gear assembly to the mattress system, further preventing side to side rotation of the head of an infant during transport. The straps can be affixed using any suitable attachment system, including hook and loop, e.g., VELCRO®, snaps, buckles, e.g. side release, e.g., FASTEX®, or other attachment systems known in the art.

### Mattresses

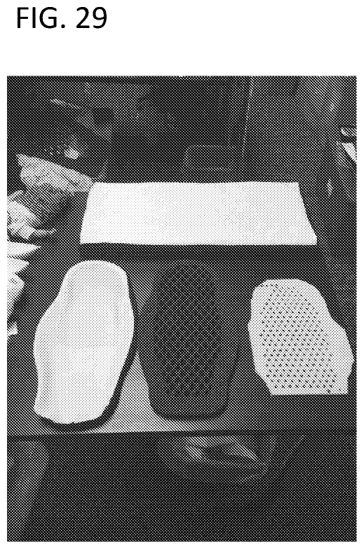

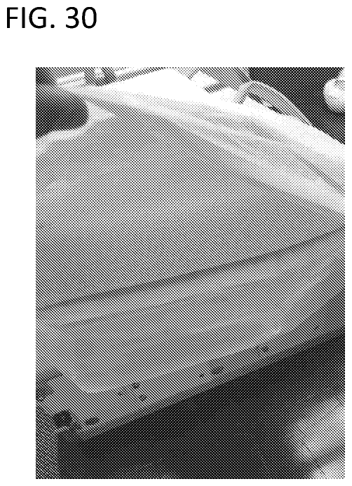

Some embodiments of the invention feature a two-component mattress system, as shown in FIG. 29. One component of the system is a traditional rectangular mattress which fits within a transport isolation tray (e.g., a transport tray having dimensions of about 24 inches by 10.5 inches, e.g. an ISOLETTE®), and the other component is a removable mattress. The removable mattress may have dimensions of about 9 inches by 16 inches, (e.g., 5 inches by 12 inches, 6 inches by 13 inches, 7 inches by 14 inches, 8 inches by 15 inches, 9 inches by 16 inches, 10 inches by 17 inches, 11 inches by 18 inches, or 12 inches by 19 inches) and may be designed to be added for the care of a smaller infant. Alternatively, the rectangular mattress may be used as a standalone mattress for transporting an infant in the transport isolation tray. When used as a standalone mattress, the rectangular mattress may have dimensions of about 11 inches by 24 inches, (e.g., 7 inches by 20 inches, 8 inches by 21 inches, 9 inches by 22 inches, 10 inches by 23 inches, 11 inches by 24 inches, 12 inches by 25 inches, 13 inches by 26 inches, or 14 inches by 27 inches). The two mattresses may be placed together, e.g., by stacking the removable mattress on top of the rectangular mattress. In another embodiment, the rectangular mattress contains a cutout in the center which is shaped to receive the removable mattress such that they nestably engage. In this configuration, the peripheral edges of the removable mattress contact the edges of the cutout in the rectangular mattress (e.g., the peripheral mattress) such that the removable mattress is able to resist lateral motion during a transportation event. The mattress system, e.g., the rectangular mattress and the removable mattress, may be made of a suitable foam material, such as a viscoelastic foam, e.g., polyurethane, e.g., memory foam. Other suitable foams for mattresses are known in the art. The mattresses may be made of a single type of foam, e.g., both be made of polyurethane, or alternatively, each can be made of a different foam material to allow the compressibility of the mattress system to be adjusted to suit the infant being transported. For example, the foam the mattress is made from can be chosen to have a slow or fast recovery time after compression to control the level of support provided to the infant during transport. The mattresses may be constructed of a single layer of foam, or alternatively, may be made of multiple layers, e.g., 2 or more, to reach the desired thickness or compressive properties. An example of a multilayered mattress is shown in FIG. 30. Each of the layers may be made of the same foam, each layer may be a different foam, or may be different combinations of foam. In another embodiment of the invention, the mattresses whether used as a system or standalone, may be made of different types of foam on either side such that the both sides of the mattress can be used for transporting an infant. For example, one or both of the mattresses, e.g., the removable mattress or the rectangular mattress, may be made of a quick release foam on one side and a slow release foam on the opposite side, allowing the mattress to be used with a range of different sized infants. When configured as a dual sided mattress, a smaller infant, such as one with a body mass between about 500 grams to about 1500 grams, may be secured to a side of the mattress made of a slow release memory foam. When the need arises to transport a larger infant, the mattresses may be flipped over to the other side, which may be made of a faster release foam sufficient to support an infant of a body mass between about 1500 grams to about 4500 grams.

In such embodiments in which a removable mattress is nestably engaged in a peripheral mattress, the peripheral mattress may be of any suitable material composition to resist lateral motion and need not necessarily be the same material composition as the removable mattress or even be of a material suitable to directly support the infant thereupon.

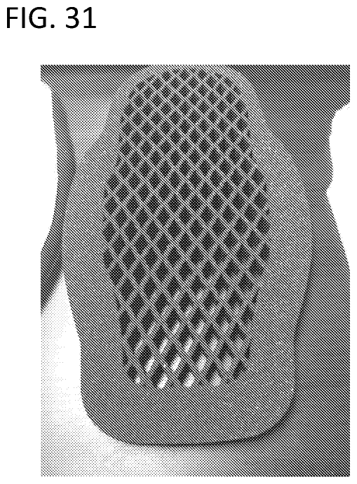

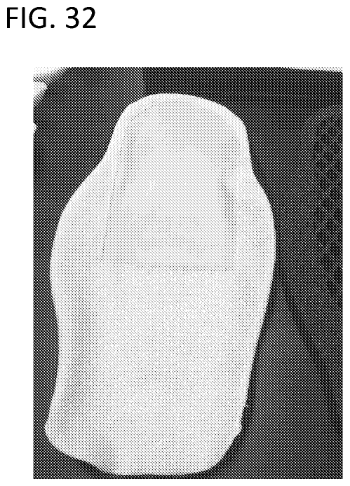

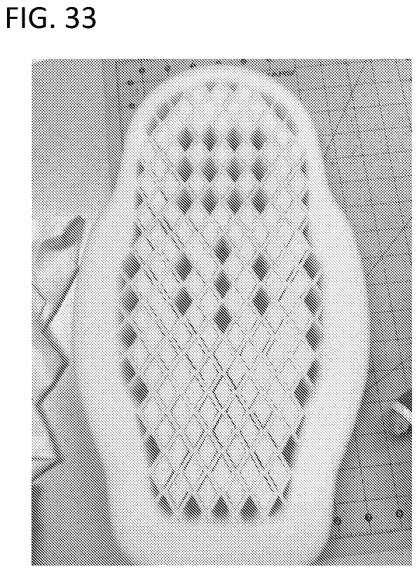

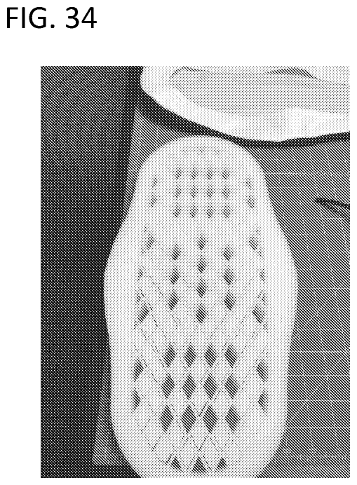

The removable mattress may be a solid piece of foam or may be made to have conduits throughout its structure (e.g., a plurality of conduits running vertically throughout). Examples of mattresses with conduits in them are shown in FIGS. 31-36. Such conduits can be incorporated by any suitable means known in the art or described herein. For example, in some embodiments, the conduits in the mattresses are drilled out or molded therein. The conduits can be of any suitable shape, such as diamond (FIGS. 30-34 and FIG. 36), circular (FIG. 35), or any polygonal shape. The conduits in the removable mattress may be left open, or may be selectively filled with a removable insert (e.g., having the shape of the conduit). For example, FIGS. 33 and 34 show uncovered removable mattresses with diamond shaped conduits having selected conduits filled in with diamond-shaped foam inserts. The pattern of conduits that are filled exerts control over the compressive and thermal properties of the mattress.

The conduits formed in the removable mattress and the removable inserts are configured to serve two purposes. The first purpose is to allow for the customization of the support in a localized region of the removable mattress. When the removable inserts are not installed in the conduits, the open pattern will compress more, providing less support to a specific region of the infant. When the removable inserts are installed in the conduits, the increase in the material provides a greater level of support to the infant. The use of the removable inserts allows the clinical to tailor the compressive modulus of the mattress for a specific infant or a specific region of the body of an infant based on a physical parameter of the infant, such as body mass or body size. The second purpose of the conduits and removable inserts of the removable mattress is to permit heat to be transferred more evenly to and from the baby by controlling where heat can be passed through the material. These inserts can be reconfigured to provide locations on the mattress where more heat (e.g., removal of the insert) or less heat (e.g., use of the insert) is needed for the infant during transport.

The relative area or volume of the conduits (e.g., conduits not occupied by removable inserts) relative to the area or volume of mattress foam will depend on the specific application of the removable mattress, e.g., the specific size and body mass of the infant, the area of the infant's body needing more or less support, and/or the level of thermal conduction needed to stabilize the infant's body temperature. The relative surface area (e.g., lateral surface area, e.g., top surface area) that is open as a conduit may be from 1% to 70% (e.g., from 2% to 60%, from 5% to 50%, or from 10% to 40%, e.g., from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, or from 65% to 70%, e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, or about 70%). The relative volume within the mattress that is open as a conduit may be from 1% to 70% (e.g., from 2% to 60%, from 5% to 50%, or from 10% to 40%, e.g., from 1% to 5%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, or from 65% to 70%, e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, or about 70%).

Similarly, the number of removable inserts installed in the removable mattress at any time will depend on the specific application of the removable mattress, e.g., the specific size and body mass of the infant, the area of the infant's body needing more or less support, and/or the level of thermal conduction needed to stabilize the infant's body temperature. The removable mattress may be used with anywhere from 0% to 100% of the removable inserts, e.g., 0%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of the removable inserts. An additional feature of the conduits made through the mattresses is for safety. By having the conducts throughout the mattresses, the infant is able to breathe normally in the event that infant should manage to flip face down into the mattress.

The conduits may have a median cross-sectional width or diameter from about 0.1 cm to 5 cm (e.g., from 0.2 cm to 4 cm, from 0.5 cm to 3 cm, or from 1 cm to 2 cm, e.g., about 0.1 cm, about 0.2 cm, about 0.3 cm, about 0.4 cm, about 0.5 cm, about 0.6 cm, about 0.7 cm, about 0.8 cm, about 0.9 cm, about 1 cm, about 1.5 cm, about 2 cm, about 2.5 cm, about 3 cm, about 3.5 cm, about 4 cm, about 4.5 cm, or about 5 cm).

In addition to the conduits, the removable mattress may further include channels that propagate laterally, e.g., horizontal, e.g., in the plane that an infant would be oriented on, throughout its structure. The channels formed in the removable mattress are configured to serve at least two purposes. One purpose of the channels in the removable mattress is to permit heat to be transferred more evenly to and from the baby by controlling where heat can be passed along the length. The channels, being in the lateral plane of the mattress, allow for precise and even heat distribution from a heating source that is disposed below the mattress, such as a radiant element, e.g., a Peltier element, or a source of light. The width and depth of the channels in the removable mattress are able to control the heat distributed in the mattress by both changing the surface area that is contacted by the heat source as well as controlling the air flow below the removable mattress.

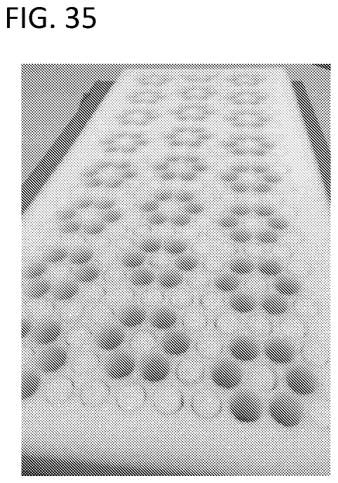

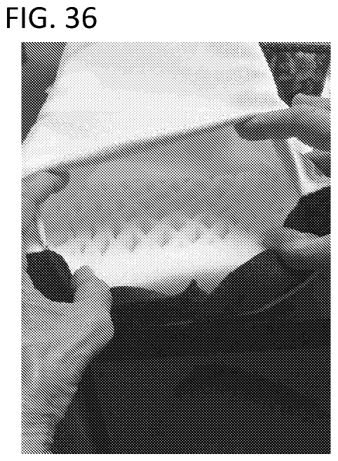

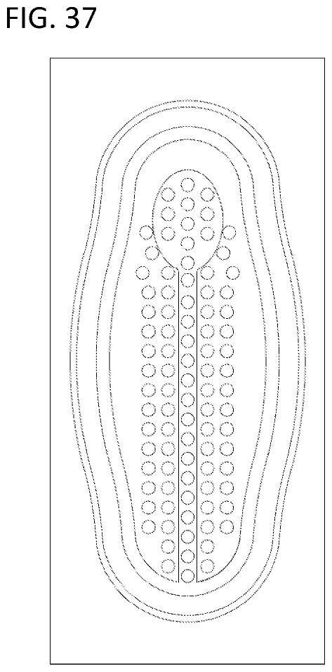

Another purpose of the channels in the removable mattress is to provide areas of localized support for the body of the infant. The forming of channels in the defines areas in the removable mattress, such as shown in FIG. 37, where the body of an infant is supported, such as the upper and lower torso and legs. In some embodiments, the conduits are positioned to provide support for infants having a range of body weights. For example, a mattress may have conduits configured to support infants having a body weight in the range from about 1,500 g to about 5,000 g (e.g., from about 1,500 g to about 2,000 g, from about 2,000 g to about 2,500 g, from about 2,500 g to about 3,000 g, from about 3,000 g to about 3,500 g, from about 3,500 g to about 4,000 g, from 4,000 g to about 4,500 g, or from about 4,500 g to about 5,000 g, e.g., about 1,500 g, about 1,600 g, about 1,700 g, about 1,800 g, about 1,900 g, about 2,000 g, about 2,100 g, about 2,200 g, about 2,300 g, about 2,400 g, about 2,500 g, about 2,600 g, about 2,700 g, about 2,800 g, about 2,900 g, about 3,000 g, about 3,100 g, about 3,200 g, about 3,300 g, about 3,400 g, about 3,500 g, about 3,600 g, about 3,700 g, about 3,800 g, about 3,900 g, about 4,000 g, about 4,100 g, about 4,200 g, about 4,300 g, about 4,400 g, about 4,500 g, about 4,600 g, about 4,700 g, about 4,800 g, about 4,900 g, or about 5,000 g.

A removable mattress of a mattress system of the invention may include mattress channels, mattress conduits, or both.

The channels may have a median cross-sectional width from about 0.1 cm to 10 cm (e.g., from 0.2 cm to 8 cm, from 0.5 cm to 6 cm, from 1 cm to 4 cm, or from 2 cm to 3 cm, e.g., about 0.1 cm, about 0.2 cm, about 0.3 cm, about 0.4 cm, about 0.5 cm, about 0.6 cm, about 0.7 cm, about 0.8 cm, about 0.9 cm, about 1 cm, about 1.5 cm, about 2 cm, about 2.5 cm, about 3 cm, about 3.5 cm, about 4 cm, about 4.5 cm, about 5 cm, about 5.5 cm, about 6 cm, about 6.5 cm, about 7 cm, about 7.5 cm, about 8 cm, about 8.5 cm, about 9 cm, about 9.5 cm, or about 10 cm).

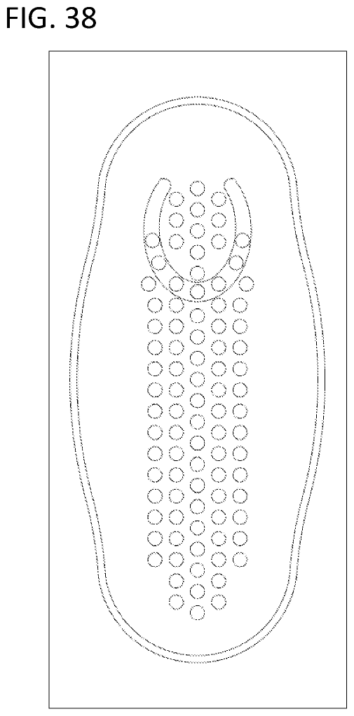

In some cases, the removable mattress may include one or more support structures for standardizing the position of the head of the infant when placed on the mattress. In particular, the support structure may be configured to maintain the head of an infant in a midline position independent of the size of the infant, e.g., 88° for an infant with a body mass of about 1,500 g to about 5,000 g. The support structure can be horseshoe shaped and elevated from the surface of the removable mattress, as shown in FIG. 38. In this embodiment, the horseshoe shaped support structure has conduits running orthogonal to the plane of the infant to provide both thermal control and/or additional support, e.g., the conduits can be covered with a correspondingly shaped member to adjust the temperature or level of support as needed. The horseshoe shaped support structure may be a permanent component of the removable mattress, e.g., integral to the removable mattress or connected using a substantially permanent connection, e.g., a chemical adhesive. Alternatively, the horseshoe shaped support structure may be releasably connected to the removable mattress such that its location may be adjusted to suit the particular dimensions of an infant. The releasable connection between the horseshoe shaped support structure and the may be any suitable attachment system, including hook and loop (e.g., VELCRO®), snaps, buttons, pins, or other suitable attachment mean known in the art.

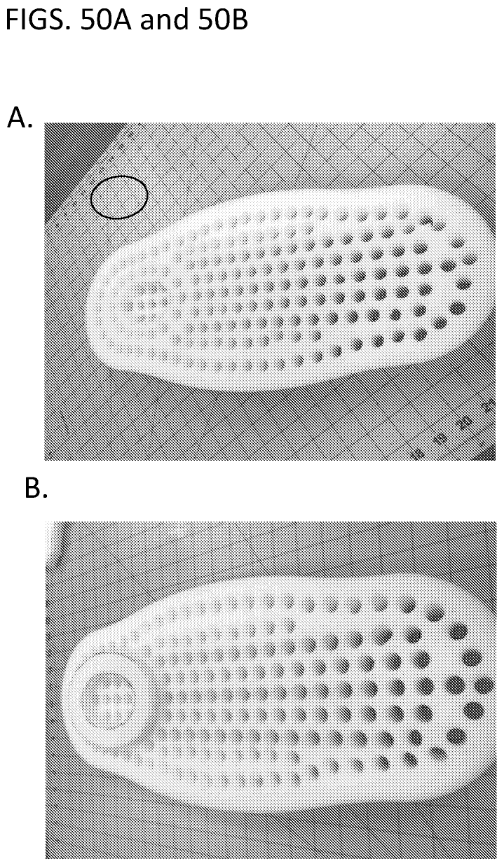

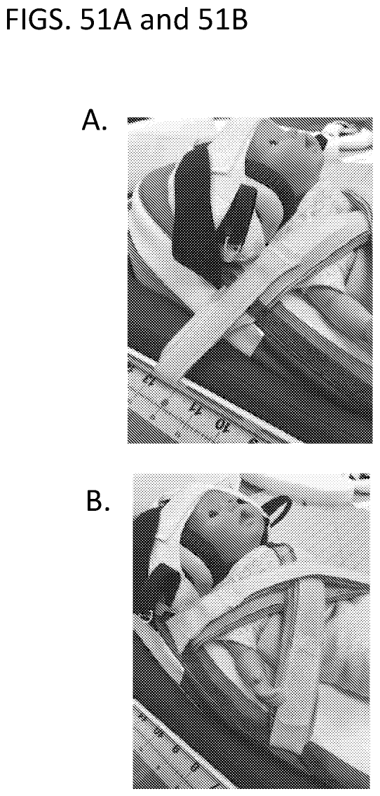

In another embodiment, the mattress is configured to accommodate smaller infants on one side and larger infants on the other side. One such embodiment, as shown in FIGS. 50A (top view) and 50B (bottom view), the mattress features a support structure on the top and bottom, wherein the support structures are different sizes and/or shapes configured to support the head of different sized infants. In one embodiment, the top of the mattress features a depression in which the head of a small infant (relative to the size of the infant configured to be supported by the other side) is configured to rest. The bottom of the mattress features a ring in which the head of a larger infant is configured to rest (when the mattress is flipped).

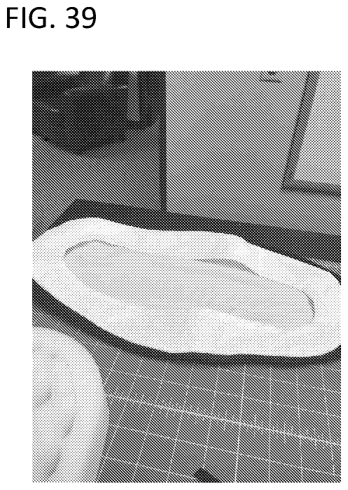

In some embodiments, the mattresses (both rectangular/peripheral and removable) are covered in one or more protective materials to enhance the comfort of the infant, provide additional vibration dampening, and to facilitate easier cleaning and maintenance. For example, the mattresses can be contained inside a cover made from a composite fabric material, such as FABRIFOAM®, or a blended fabric, e.g., cotton-LYCRA®, which may provide breathability, gentle stretching, and comfort. An example of a mattress cover is shown in FIG. 39. Additionally, they may have an additional cover made from a material which is requires little effort to maintain, such as a polyvinyl chloride or polyethylene vinyl acetate. Other low-maintenance materials for use in mattress covers are known in the art. The covers for the mattresses may be on a single side of the mattress, e.g., the side that contacts the infant during transport, or may cover the mattress in its entirety.

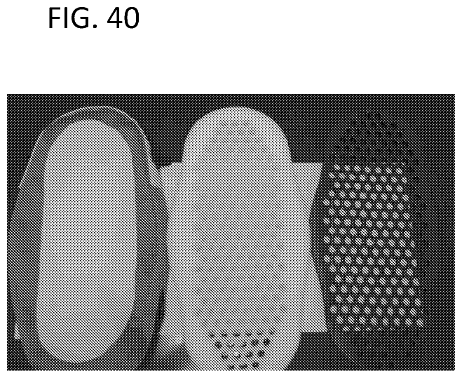

In some cases, the mattresses of the invention, when placed into a protective fabric cover may further include a plate disposed below the mattress, e.g., the mattress sits on top of the plate. This plate provides additional support and structure to the mattress for aid in secure transport. An example of a plate is shown in FIG. 40. In FIG. 40, an embodiment of a removable mattress of the invention is disassembled, showing the outer fabric cover, the mattress, and the plate (L to R in FIG. 40). In this embodiment, the plate is shaped substantially the same as the mattress and the outer cover. The plate may be of any suitable thickness such that it can provide adequate support for the mattress. The plate may be made from any suitable rigid material including metals or polymers such as acrylonitrile butadiene styrene, polytetrafluoroethylene, polyethylene terephthalate, polypropylene, e.g., corrugated plastic, polyether ether ketone, polyoxymethylene, fluorinated ethylene propylene, perfluoroalkoxy, ethylene tetrafluoroethylene, polyvinylidene fluoride, or copolymers thereof. As with the removable mattress, the plate may contain an array of conduits running vertically through its material to allow for heat transfer from the heat source of the incubation chamber, and may have less than, substantially the same, or more conduits through its structure than the mattress. An additional feature of the conduits made through the wedge, like the conduits in the mattresses of the invention, is for safety. By having the conducts throughout the plate, the infant is able to breathe normally in the event that infant should manage to flip its body over and become oriented face down into the mattress and the subsequent plate below the removable mattress. Alternatively, the plate may not have conduits if used in a location where either heat transfer and/or safety is less of a concern, e.g., when placed underneath the outer rectangular mattress. Conduits in the plate may be oriented to match the shape, size, and/or position of the conduits in the mattress, e.g., to facilitate efficient air and/or heat transfer.

### Wedge

In some embodiments, the mattress system may include a wedge configured to provide an incline to all or a portion of the removable mattress in order to elevate the infant being transported to a medically necessary position. The head of an infant should be elevated 15-30° above the 0° plane, e.g., above the surface of the mattress when the mattress is level. The wedge is configured to be placed below the removable mattress such that the removable mattress rests on top of the wedge. In another embodiment, the angle of inclination provided by the removable mattress may be integral to the removable mattress itself, e.g., the angle is fixed into the material of the removable mattress.

The size, shape, and angle of inclination provided by the wedge will be determined by the size and shape of the removable mattress (including the size, shape, and depth of the cutout in the peripheral/rectangular mattress) and the angle of inclination required. The wedge should be thick enough to support a compressive load but also be thin enough such that when installed in the mattress system, the removable mattress nestably engaged with it is still able to resist lateral motion, e.g., the peripheral edges of the removable mattress are in sufficient contact with the edges of the cutout in the peripheral/rectangular mattress). The angle of inclination provided by the wedge, when installed in the mattress system, may be can be anywhere from about 0° to about 30°, e.g., about 0°, about 1°, about 2°, about 3°, about 4°, about 5°, about 6°, about 7°, about 8°, about 9°, about 10°, about 15°, about 20°, about 25°, or about 30°, e.g., about 0° to about 10°, about 5° to about 15°, about 10° to about 20°, about 15° to about 25°, or about 20° to about 30°. An exemplary wedge that provides a 15° incline is shown in FIGS. 37 and 38. As with the removable mattress, the wedge may contain an array of conduits running vertically through its material to allow for a variable compressive modulus and/or heat transfer ability. The wedge may be made of, wholly or partially, a suitable foam material, such as a viscoelastic foam, e.g., polyurethane, e.g., memory foam, and may be the same or a different material that the mattresses. The wedge may be made of a single type of foam, e.g., made from polyurethane, or alternatively, be made of different types of foam materials to provide variable compressive properties. For example, the material the wedge is fabricated from may be chosen to be relatively uncompressible such that the angle of inclination provided by the wedge remains relatively constant while under a compressive load.

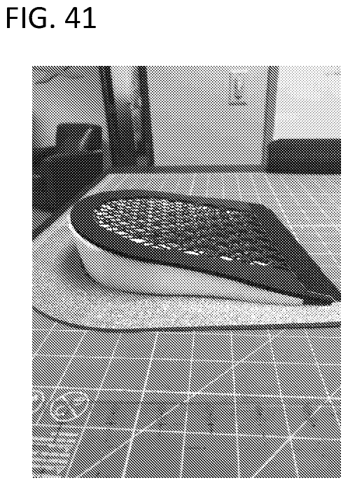

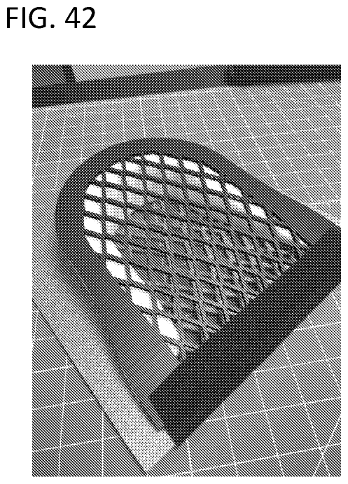

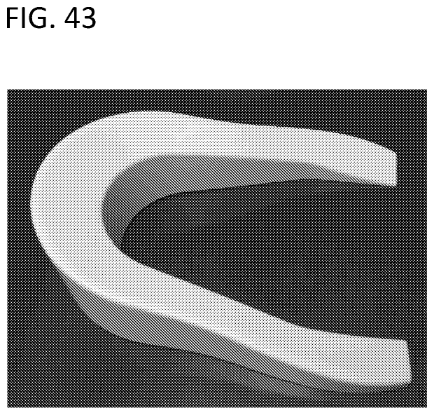

The wedge may be made from a solid block of foam, or alternatively, the wedge may be a horseshoe-shaped piece of foam having a plate disposed on top of the foam, such as the embodiment shown in FIGS. 41 and 42. In this configuration, the plate rests on top of the horseshoe-shaped piece of foam, and when engaged with a removable mattress, the removable mattress rests on top of the plate. The plate may be made of any suitable rigid material including metals or polymers such as acrylonitrile butadiene styrene, polytetrafluoroethylene, polyethylene terephthalate, polypropylene, e.g., corrugated plastic, polyether ether ketone, polyoxymethylene, fluorinated ethylene propylene, perfluoroalkoxy, ethylene tetrafluoroethylene, polyvinylidene fluoride, or copolymers thereof. As with the removable mattress, the plate used to form a wedge may contain an array of conduits running vertically through its material to allow for heat transfer from the heat source of the incubation chamber. An additional feature of the conduits made through the wedge, like the conduits in the mattresses of the invention, is for safety. By having the conducts throughout the wedge, the infant is able to breathe normally in the event that infant should manage to flip its body over and become oriented face down into the mattress and the subsequent wedge below the removable mattress. Alternatively, the wedge may be a solid piece of a substantially non-compressible, e.g., rigid, material. In this configuration, a wedge may be formed from a metal or a polymer such as acrylonitrile butadiene styrene, polytetrafluoroethylene, polyethylene terephthalate, polypropylene, e.g., corrugated plastic, polyether ether ketone, polyoxymethylene, fluorinated ethylene propylene, perfluoroalkoxy, ethylene tetrafluoroethylene, polyvinylidene fluoride, or copolymers thereof. An example of a wedge made from a solid piece of a rigid polymer is shown in FIG. 43.

In order to reduce the likelihood of the wedge being accidentally removed from the mattress system or to reduce propagation of shock or vibration, the outer perimeter of the wedge may contain a thin piece of material sized to extend out from the bottom of the wedge from about 1 inch to about 6 inches from the largest outer dimension of the wedge. An example of this thin piece of material attached to the bottom the wedge is shown in FIGS. 37 and 38. This piece of material may be integrally formed in to the wedge or may be releasably attached to the wedge. In this configuration, when the peripheral mattress (e.g., the rectangular mattress having a cutout the removable mattress) is placed on top of the wedge, the thin piece of material attached to the bottom the wedge makes contact with the peripheral mattress, preventing it from being lifted away from the peripheral mattress. The thin piece of material may be a solid piece of material, or alternatively, may have cutouts in it that match the pattern of the vertical conduits of the wedge such that heat transfer from the isolation chamber is not impeded. Alternatively or additionally, the underside of the wedge may include one or more skid-resistant features, such as a polymer pad, to contact a hard surface and prevent it from slipping during transport.

### Sources of Light

In some cases, the mattress system includes one or more sources of light. The one or more sources of light may be configured for various medical purposes, such as phototherapy, thermal regulation, or visualization. For example, premature or neonatal infants diagnosed with jaundice, e.g., yellowing of the skin or whites of the eyes from an excessive concentration of bilirubin, may be treated by blue light phototherapy. Blue light, e.g., 400 to 500 nm range of the visible portion of the electromagnetic spectrum, can be used to treat jaundice, as exposure to blue light, in particular 460 nm light, breaks down bilirubin. The source of light may also be configured to assist with visual measurements of the infant in the mattress system. For example, the source of light may be a white light, and can help a clinician visualize specific parts of the body of the infant, such as veins and arteries.

The sources of light, e.g., blue light or white light, in the mattress system may be any suitable type of light source, such as a forward biased diode, e.g., a light emitting diode (LED), that is appropriately sized and safe to use around fabric and foam materials, e.g., to reduce to risk of fire. Other appropriate sources of light are known in the art. The light source may also be of varying intensity such that the amount of light delivered to the infant can be controlled by the clinician to reduce the risk of overexposure, which may be monitored by a photodetector, e.g., a charge-coupled device (CCD) or reverse bias LED, installed within the mattress system or the isolation chamber. The intensity of the light may be controlled by a rheostat or other similar current limiting device. In some cases, the one or more sources of light may contain sources of light with the same output wavelength; alternatively, the one or more sources of light may be individual light sources for different regions of the electromagnetic spectrum that may be independently controllable. As a non-limiting example, the source of light may be a strip of LEDs that contains both blue light and white light LEDs, where the LEDs that are of a particular wavelength are independently controllable, e.g., the blue light LEDs can be controlled separately from the while light LEDs.

Each of the one or more sources of light may contain any suitable number of individual light sources, e.g., from 1 individual source of light to 100 individual sources of light (e.g., from 1 to 100 individual sources of light, from 5 to 90 individual sources of light, from 10 to 80 individual sources of light, from 15 to 70 individual sources of light, from 20 to 60 individual sources of light, from 25 to 50 individual sources of light, or from 30 to 40 individual sources of light, e.g., 1 individual source of light, 2 individual sources of light, 3 individual sources of light, 4 individual sources of light, 5 individual sources of light, 6 individual sources of light, 7 individual sources of light, 8 individual sources of light, 9 individual sources of light, 10 individual sources of light, 15 individual sources of light, 20 individual sources of light, 25 individual sources of light, 30 individual sources of light, 35 individual sources of light, 40 individual sources of light, 45 individual sources of light, 50 individual sources of light, 55 individual sources of light, 60 individual sources of light, 65 individual sources of light, 70 individual sources of light, 75 individual sources of light, 80 individual sources of light, 85 individual sources of light, 90 individual sources of light, 95 individual sources of light, or 100 individual sources of light).

The one or more sources of light may be at any practical location in the mattress system for the intended purpose of a particular light source. In some cases, the one or more sources of light may be located underneath a mattress of the mattress system of the invention (e.g., on or near a plate), on one or more sides of a mattress of the mattress system of the invention, or a combination of both. The one or more sources of light at each location in the mattress system of the invention may all be at the same wavelength. Alternatively, one or more sources of light at each location in the mattress system of the invention may each output a different wavelength of light, and which wavelength of light to be used at a specific location may be determined by the clinician.

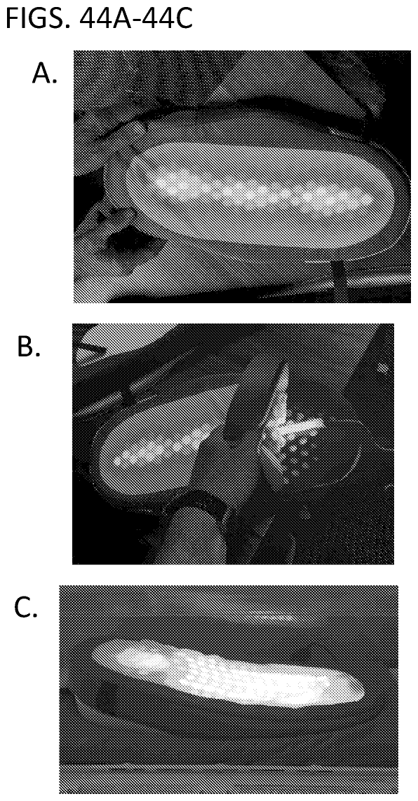

As an example, one or more sources of light may be installed underneath the wedge described herein or on one or more components of the mattress system described herein. For example, a source of blue light may be installed underneath a mattress to provide phototherapy, e.g., for treating jaundice. An embodiment of a peripheral mattress including a source of light is shown in FIGS. 44A and 44B. In this embodiment, the source of light is a rod containing a plurality of blue LEDs that illuminate underneath the peripheral mattress, as shown in FIG. 44A. FIG. 44B shown the rod of LED installed inside the peripheral mattress between the mattress and the plate.

In some cases, a component of a mattress system of the invention, e.g., a peripheral mattress, may include two or more sources of light. For example, a source of light, e.g., blue light, e.g., blue LEDs, may be installed underneath a mattress (as described in FIGS. 44A-44B), and one or more sources of white light may be installed on the one or more sides of a mattress to provide illumination for installing medical devices or for visual monitoring of the infant. The one or more sources of light may be attached substantially permanently to the installation location on the mattress, e.g., using a chemical adhesive or sewn on. Alternatively, the one or more sources of light may be releasably attached to the installation location on the mattress, e.g., hook and loop (VELCRO®), hook and eye, snaps, or placed into a pocket sewn into the fabric covering of a mattress system component.

The shape of the one or more sources of light may be of any suitable shape, such as round, oval oblong, tubular, etc. A skilled artisan can appreciate that the location, number of individual sources of light required, and the intended use will have a role in determining the shape of the one or more sources of light.

In some embodiments, three parallel strips of light (e.g., blue light) are provided in a longitudinal configuration within the mattress. For example, the mattress shown in FIG. 44C contains three parallel blue strips, i.e., diodes, wherein the two lateral strips are about two thirds the length of the central strip.

In some embodiments, the invention features multiple sources of light arranged within or around the mattress in a manner configured to provide a constant intensity of light over all or a portion of the surface. For example, in some embodiments, the light intensity varies by less than 60%, less than 50%, less than 40%, less than 30%, less than 20%, less than 10%, less than 8%, less than 5%, or less than 2% across all or a portion of the upper surface of the mattress. In some embodiments, the portion of the mattress that has the consistent light intensity is from 10%-90% of the upper surface of the mattress (e.g., from 20%-80%, from 30%-70%, or from 40%-60%).

In some embodiments, the mattress can be covered (e.g., the upper surface and/or lower surface) in a material that enhances the consistency of light exposure provided by the one or more light sources. For example, the fabric can be any suitable composition and/or weave to enhance diffraction and/or reflection of the light emitted from the light source. In some embodiments, the mattress and/or cover is configured to have a suitable thickness between the light source and the infant to facilitate suitable intensity of light to contact the infant (e.g., between 0.5 and 2 inches in an uncompressed configuration, e.g., from 1 inch to 1.8 inches, e.g., about 1.5 inches)).

The one or more sources of light may be configured to further act as a source of heat for controlling the temperature of the local environment around the infant. The amount of heat produced by the one or more sources of light will depend on the type of light source used, the output wavelength of the one or more sources of light, the level of control of the one or more sources of light, e.g., controllable using a rheostat, and the total number of individual sources of light configured to act a source of heat. The heat produced by the one or more sources of light can directly heat the infant, e.g., the infant is exposed to the one or more sources of light by direct contact; alternatively, the heat produced by the one or more sources can indirectly heat the infant. For example, the one or more sources of light may cause in increase in one or more components of the mattress system that directly contacts the infant. Alternatively, the combination of the conduit and/or channels in one or more of the components of the mattress system and the heat produced by the one or more sources of light may allow for warm air to be effectively circulated around the infant. In some instances, the one or more sources of light may work in combination with sources of light that are configured to act as a source of heat. As a non-limiting example, a source of blue light may be installed underneath a mattress to provide heat to the infant, and one or more sources of white light may be installed on the one or more sides of a mattress to provide illumination for installing medical devices or for visual monitoring of the infant.

In some cases, the one or more sources of light may be configured to act as visual indicators for sensors connected to one or more components of the mattress system. The mattress system of the invention may have various sensors installed within one or more components to monitor ambient conditions of the infant, e.g., skin temperature, interior temperature and humidity of the incubation chamber, or level of oxygen. Coupled to an appropriate sensor for a specific condition, one or more sources of light may provide a real-time indicator of said condition. As a non-limiting example, when coupled to a thermal sensor, e.g., a thermocouple or a thermistor, one or more sources of light may emit light of a certain wavelength to indicate a pre-defined temperature alarm. In this configuration, when a measured temperature is above the pre-defined level, one or more sources of light may emit a red light to alert a clinician; alternatively, when a measured temperature is below the pre-defined level, one or more sources of light may emit a different wavelength, such as blue, of light to alert a clinician. The one or more sources of light configured to act as an indicator may indicate by presenting a constant output, e.g., “always on” or may alternate between off and on, e.g., blinking or flashing, and remain in the alert state until the condition that triggered the alert is remedied. A range of parameters that can be usefully indicated using a light source of the present invention will be readily apparent to a skilled artisan in view of the present disclosure.

The source of power for the one or more sources of light may be from a standard wall receptacle connected to a domestic power grid, e.g., 120 V 60 Hz AC power, or may come from a portable source of electricity, e.g., a battery, when not connected to a domestic power grid, e.g., in a remote field location.

### Infant Pod

The mattress system of the current invention may be configured for use with any commercially available radiantly warmed transport isolation system, including but not limited to, the ISOLETTE® and CALEO® from Dräger, the GIRAFFE® from GE, the NATALCARE LX® from Natus, the BABYGUARD® from Dixon, the POLYTREND® and BABY SHUTTLE® from Ginevri, the THERMOCARE VITA® from Weyer, and others that are commercially available. The mattress system of the invention may further be configured for use in low-cost field-deployable neonatal incubation chambers that restrict evaporation and heat loss, such as the LIFERAFT® Infant Incubator or a double-walled polymer bag placed around certain portions of the infant's body.

A skilled artisan can appreciate the straightforward modifications to the mattress system of the invention to accommodate different transport isolation systems, such as exterior shape or exterior dimensions, e.g., length or width, or by providing a suitable adapter, e.g., a foam spacer, to make up the difference in one or more dimensions between the mattress system of the invention and the commercial transport isolation system. When used with an appropriately shaped rectangular mattress or an appropriately shaped spacer, the mattress system of the present invention may be used with a radiantly warmed transport isolation system that is in any location that an infant is born, whether the infant is to be transported or not. In addition, the use of an appropriately shaped rectangular mattress or an appropriately shaped spacer with an available radiantly warmed transport isolation system reduces the handling and unintended stimulation to the infant, e.g., the infant would not have to be transferred between multiple transport systems, increasing the stability and safety of the infant.

### Methods

Featured herein are methods for securing the position of an infant using one or more support rolls described herein. In particular, the methods include contacting the infant with one more support rolls to elevate a first portion of the infant's body relative to a second portion of the infant's body. The first portion of the infant's body may be chosen to be lateral to the infant's midline, superior to the infant's waist, or inferior to the infant's waist. The support rolls may be used to provide an elevation up to about 45° of the first portion of the infant's body relative to the second portion of the infant's body. The support rolls may further be used to elevate a third portion of the infant's body relative to the second portion of the infant's body.

The invention also provides methods of reducing shock or vibration to an infant during transportation using a mattress system described herein by securing the infant to the mattress system using a plurality of straps. Before the straps are secured down, one or more body parts of the infant may be elevated using one or more support rolls described herein placed on the infant's body in the appropriate location. While the infant's body is secured to the mattress system and specific support rolls are supported with one or more support rolls, the infant's ears may be contacted by a head gear assembly described herein configured to reduce the exposure to noise during transport.

The invention further provides a method of reducing shock, vibration, or noise exposure to an infant during transportation by providing a mattress system described herein, further providing a head gear assembly described herein, and securing both the head gear assembly to the infant and securing the infant to the mattress system. Before the infant is secured to the mattress system, one or more body parts of the infant may be elevated using one or more support rolls described herein placed on the infant's body in the appropriate location.

The invention additionally provides a method of reducing shock or vibration while providing elevation to specific portions of an infant during transportation by providing the mattress system described herein, further providing a plurality of support rolls described herein, and securing the infant to the mattress system using the plurality of straps of the mattress system. While the infant's body is secured to the mattress system and specific support rolls are supported with one or more support rolls, the infant's ears may be contacted by a head gear assembly described herein configured to reduce the exposure to noise during transport.

Further provided are methods of reducing the risk of an intraventricular hemorrhage (IVH) and/or PPHN to an infant during transportation using a mattress system of the invention. IVH may result from excessive left-to-right (e.g., side-to-side) motion of the head, and is more prevalent in very small infants. In particular, the invention provides method of reducing the risk of IVH and/or PPHN by providing the mattress system described herein, providing the head gear assembly described herein, further providing a plurality of support rolls described herein, and securing the infant to the mattress system using the plurality of straps of the mattress system.

### EXAMPLES

### Example 1—Securing an Infant in a Mattress System of the Invention

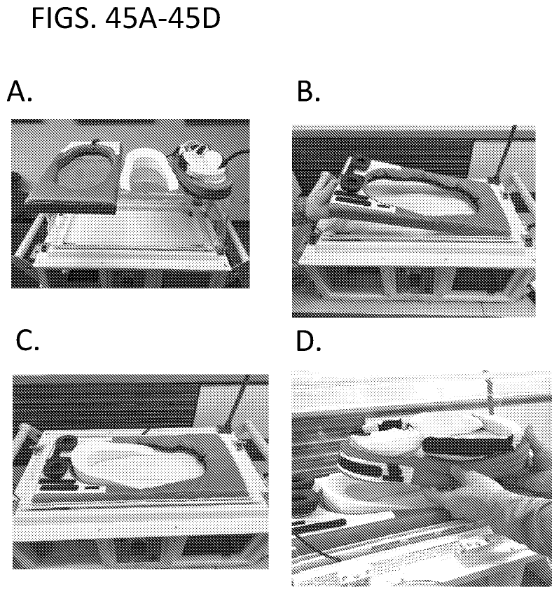

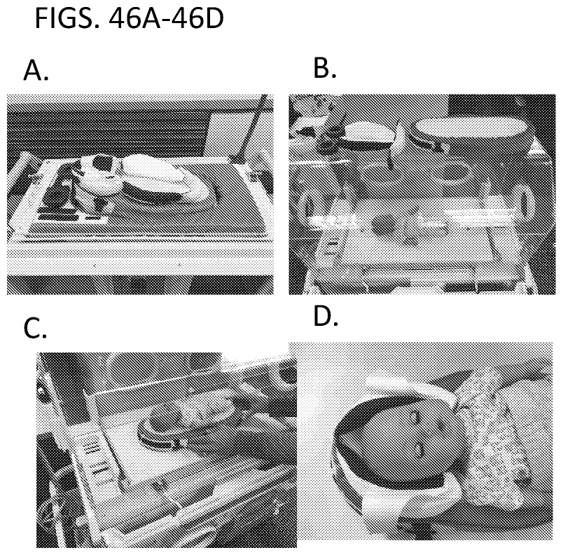

FIGS. 45A-48E provide a series of images showing a method of securing an infant to an embodiment of a mattress system of the invention. As is shown in FIG. 45A, the mattress system of the invention is disassembled from its packaging and the components separated prior to departing to the outbound hospital to pick up the infant. The outer rectangular mattress, i.e., the outer Docking Station, is placed into the interior tray of the transport isolation chamber (FIG. 45B), followed by the placement of the elevation wedge, e.g., the 15° wedge, into the interior cutout of the outer rectangular mattress (FIG. 45C), then the removable mattress with the supporting components is placed on top of the wedge in the interior cutout of the outer rectangular mattress (FIG. 45D). The mattress system is now ready for patient transport (FIG. 46A).

As is shown in FIGS. 46B-46D, to prepare the infant for transport, the mattress system of the invention is disassembled and the removable mattress with the supporting components, e.g., one or more body support rolls and a hear gear assembly, are taken to the infant (FIG. 46B). The removable mattress is placed underneath the infant while maintaining connections of all attached medical devices (FIG. 46C), and the head gear assembly is positioned underneath the infant's head such that the ear protectors are disposed near each of the infant's ears (FIG. 46D). In some cases, the mattress system of the invention may include an optional multi-point, e.g., a five-point, safety harness to further secure the infant to the mattress system.

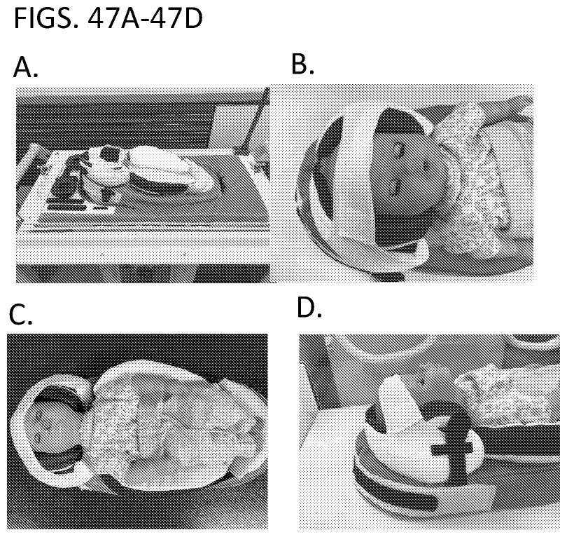

In FIGS. 47A and 47B, the ear protectors are placed over each ear, and the securing member of the head gear assembly is connected together to secure the ear protectors to the infant's head. The body support rolls, optionally including a foot roll, are placed underneath the infant's sides and feet, respectively, to position the infant in the optimal fetal flexed position (FIG. 47C). The sides of the head gear assembly is then secured to the removable mattress using a securing member (FIG. 47D).

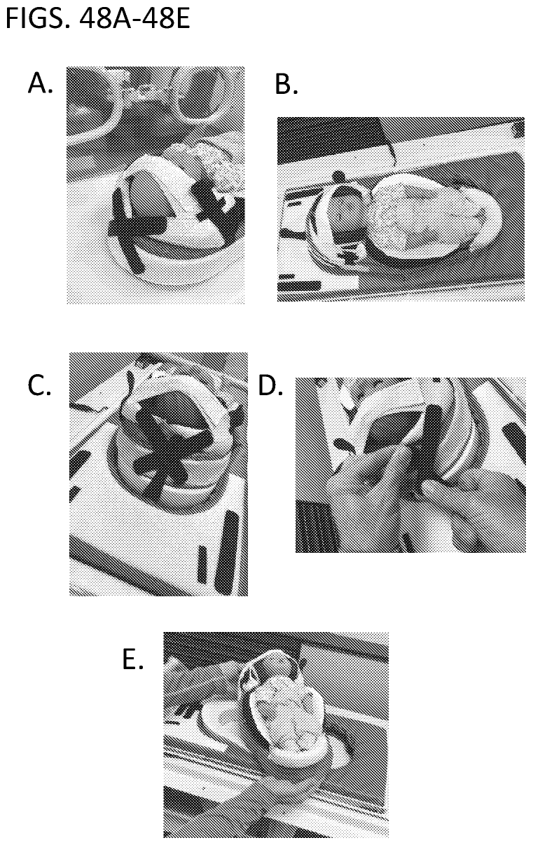

As is shown in FIG. 48A, the support rolls of the head gear assembly are then secured to the removable mattress using a securing member, e.g., in a crossed pattern, in order to keep the airway of the infant open during transport. After securing the head gear assembly to the removable mattress, the infant in the removable mattress is then placed in the outer rectangular mattress with the wedge installed (FIG. 48B) and secured using securing members (FIG. 48C). The optional safety harness, e.g., five-point safety harness, if in use, may be further secured to the transport isolation chamber. As shown in FIG. 48D, upon arrival of the infant to the destination, the infant can be removed from the transport isolation chamber by removing the securing elements holding the removable mattress the outer rectangular mattress, leaving the infant secured to the removable mattress (FIG. 48E) for localized transport within the destination.

### Example 2—Accessories for Use with the Mattress System of the Invention

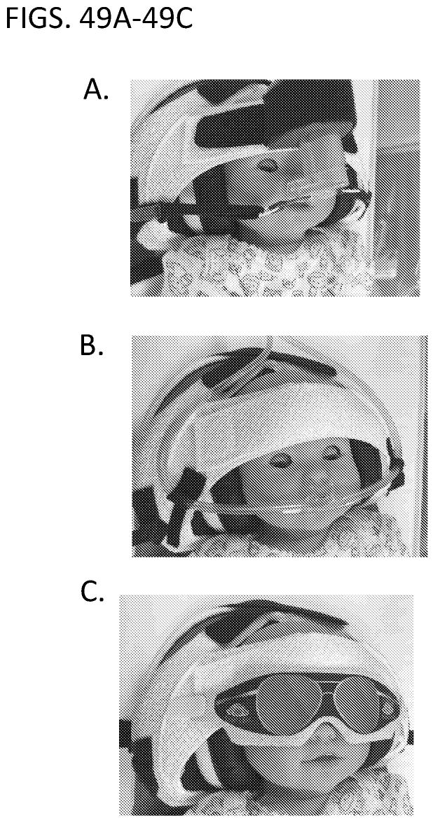

FIGS. 49A-49C show pictures of medical device accessories that may be used with the mattress system of the invention.

FIG. 49A shows a nasal Continuous Positive Airway Pressure (CPAP) device installed over the nose of an infant secured to an embodiment of a removable mattress and head gear assembly of the invention in the supine midline position. A lower portion of the mask of the CPAP device is secured to the face of the infant using a securing member that is releasably connected to a compatible securing element on the cover of the removable mattress, e.g., a strip of hook-and-loop closure material, using a separate securing element. An upper portion of the mask of the CPAP device is secured to the face of the infant using a securing member that is releasably connected to a compatible securing element on the exterior fabric material of the head gear assembly e.g., a strip of hook-and-loop closure material, using a separate securing element.

FIG. 49B shows a nasal cannula with its nares installed in the nose of an infant and secured to an embodiment of a removable mattress and head gear assembly of the invention in the supine midline position. The tubes of the nasal cannula are secured to the head gear assembly on both sides of the infant's head by securing elements that releasably connect to the fabric covering of the head gear assembly, e.g., using a hook-and-look closure material. As shown, the tubes of the cannula are connected at a position lateral to the ear protectors of the head gear assembly to keep the tubes away from sensitive facial structures and to reduce the risk of injury.

FIG. 49C shows an infant secured in an embodiment of a removable mattress and head gear assembly of the invention with the eyes covered by a protective bilishade. The bilishade is placed over the eyes of the infant with the notch aligned with the bridge of the nose. Each end of the bilishade is secured to the fabric cover of the head gear assembly by a securing element (e.g., a section of hook-and-loop closure material) that is connected to each end of the bilishades.

### OTHER EMBODIMENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure that come within known or customary practice within the art to which the disclosure pertains and may be applied to the essential features hereinbefore set forth.

What is claimed is:

1. A head gear assembly configured to immobilize a head of an infant and reduce noise exposure to the infant during transport, the head gear assembly comprising:
(a) two ear protectors, each comprising a housing having an inside recess and a perimeter, wherein the inside recess of the housing comprises a sound-absorbing material, wherein the perimeter of the housing comprises a cushioning material to contact the infant's head;
(b) a support structure having two stabilizing elements lateral to the ear protectors, the support structure configured to resist rotation of the head; and
(c) a securing element configured to exert a compressive force against the head to secure the ear protectors and the support structure to the head.

2. The head gear assembly of claim 1, wherein, when installed, all or a portion of each ear protector is between the head and one of the stabilizing elements.

3. The head gear assembly of claim 1, wherein (a), (b), and (c) are integrally formed.

4. The head gear assembly of any one of claims 1-3, wherein the securing element comprises an elastic fabric.

5. The head gear assembly of claim 4, wherein the attachment mechanism comprises a hook-and-loop attachment mechanism.

6. The head gear assembly of any one of claims 1-5, wherein the housing of the ear protector is an impact resistant material.

7. The head gear assembly of claim 6, wherein the impact resistant material comprises polytetrafluoroethylene, polyethylene terephthalate, polypropylene, polyether ether ketone, polyoxymethylene, fluorinated ethylene propylene, perfluoroalkoxy, ethylene tetrafluoroethylene, polyvinylidene fluoride, or a copolymer thereof.

8. The head gear assembly of any one of claims 1-7, wherein the sound-absorbing material comprises polyurethane, polyether, polyester, melamine, or a combination thereof.

9. The head gear assembly of any one of claims 1-8, wherein the cushioning material of the perimeter of the ear protector comprises polyurethane, polyether, polyester, melamine, a liquid cushion, a gel cushion, or a combination thereof.

10. The head gear assembly of any one of claims 1-9, further comprising one or more securing elements for an external medical device.

11. A support roll for stabilizing a position of an infant, the support roll having a wedge-shaped cross-section, wherein the support roll has a length from 6 to 24 inches, a thickness from 1 to 4 inches, and a maximum width from 3 to 6 inches.

12. The support roll of claim 11, wherein the support roll is encased in a fabric, wherein the fabric is connected to a strap having a first end and a second end, wherein the first end is connected to the fabric at a portion of the fabric contacting the vertex of the wedge and the second end is removably attachable to the fabric.

13. The support roll of claim 11 or 12, wherein the support roll is made from polyurethane.

14. The support roll of any one of claims 11-13, wherein the support roll is crescent-shaped.

15. The support roll of any one of claims 11-14, wherein the width of the strap decreases from the first end to the second end.

16. The support roll of claim 15, wherein the second end of the strap comprises an attachment mechanism.

17. The support roll of claim 16, wherein the attachment mechanism is a hook-and-loop mechanism.

18. A mattress configured to support an infant's body, the mattress comprising a top surface, a bottom surface, and a core material therebetween, wherein the core material comprises a plurality of conduits running vertically therethrough, wherein the plurality of conduits provides a variable compressive modulus over the top surface of the mattress.

19. The mattress of claim 18, wherein the mattress is a removable mattress configured to nestably engage with a peripheral mattress, wherein the peripheral mattress comprises a cutout configured to contact the periphery of the removable mattress to resist lateral motion of the removable mattress.

20. The mattress of claim 18 or 19, wherein the array of conduits enhances heat diffusivity from the bottom surface of the mattress to the top surface of the mattress.

21. The mattress of claim 20, wherein some of the plurality of conduits comprises a removable member configured to fill the conduit.

22. The mattress of any one of claims 18-21, wherein the mattress comprises viscoelastic foam, polyurethane foam, memory foam, or a combination thereof.

23. The mattress of any one of claims 18-22, wherein the mattress is covered in a protective material.

24. The mattress of claim 23, wherein the protective material comprises a composite fabric, fabric blend, vinyl, acetate, or a combination thereof.

25. The mattress of claim 23 or 24, further comprising a plate disposed below the mattress.

26. The mattress system of any one of claims 18-25, wherein the mattress is configured to bend to an incline.

27. A mattress system for secure transport of an infant, the mattress system comprising:
(a) a peripheral mattress comprising a cutout; and
(b) a removable mattress comprising a top surface, a bottom surface, and a core material therebetween having a shape configured to nestably engage within the cutout of the peripheral mattress;
wherein, upon engagement with the peripheral mattress, lateral motion of the removable mattress is reduced.

28. The mattress system of claim 27, wherein the peripheral mattress is sized to fit within the tray of an incubator chamber.

29. The mattress system of claim 27 or 28, further comprising a wedge configured to be positioned below the removable mattress.

30. The mattress system of claim 29, wherein the wedge, when installed, provides an incline to the top surface of the removable mattress.

31. The mattress system of claim 30, wherein the wedge, when installed, provides an incline of between 0 and 30° to the top surface of the removable mattress.

32. The mattress system of claim 31, wherein the wedge, when installed, provides an incline of about 15° to the top surface of the removable mattress.

33. The mattress system of claim 27 or 28, wherein the top surface of the removable mattress provides an incline of between 0 and 30°.

34. The mattress system of claim 33, wherein the top surface of the removable mattress is at an incline of about 15°.

35. The mattress system of any one of claims 27-34, further comprising a plurality of straps, each of the plurality of straps comprising a first end and a second end, wherein the first end and second end of each of the plurality of straps comprises a connector configured to secure the infant to the mattress.

36. The mattress system of any one of claims 27-35, wherein the removable mattress further comprises openings through the top surface, bottom surface, and core material for the plurality of straps to pass through.

37. The mattress system of claim 35 or 36, wherein at least one or more of the plurality of straps is configured to secure the head of the infant to the mattress system.

38. The mattress system of any one of claims 35-37, wherein at least one or more of the plurality of straps is configured to secure the torso of the infant to the mattress system.

39. The mattress system of any one of claims 35-38, wherein at least one or more of the plurality of straps is configured to secure the pelvis of the infant to the mattress system.

40. The mattress system of any one of claims 27-39, wherein the system further comprises one or more sources of light.

41. The mattress system of claim 40, wherein the one or more sources of light is a light emitting diode.

42. The mattress system of any one of claims 27-41, wherein the mattress system comprises the head gear assembly of any one of claims 1-9.

43. The mattress system of any one of claims 27-41, wherein the mattress system comprises one or more of the support rolls of any one of claims 11-17.

44. A method of stabilizing a position of an infant, the method comprising contacting the infant with one or more support rolls to elevate a first portion of the infant's body relative to a second portion of the infant's body, wherein the first portion of the infant's body is:
(a) lateral to the infant's midline;
(b) superior to the infant's waist; or
(c) inferior to the infant's waist.

45. The method of claim 44, wherein the support roll has a wedge-shaped cross-section.

46. The method of claim 44 or 45, wherein the support rolls elevate a first portion of the infant's body between an angle from about 0° to about 45°.

47. The method of any one of claims 44-46, further comprising contacting the infant with one or more support rolls to elevate a third portion of the infant's body relative to the second portion of the infant's body.

48. A method of reducing shock or vibration to an infant during transportation, the method comprising positioning the infant on the mattress system of any one claims 27-43.

49. The method of claim 48, further comprising securing the infant to the mattress system using a plurality of straps.

50. The method of claim 48 or 49, wherein the mattress system is housed within an incubator chamber.

51. The method of any one of claims 48-50, wherein a portion of the infant's body is elevated by a plurality of support rolls.

52. The method of claim 51, wherein the support rolls are the support rolls of any one of claims 11-17.

53. The method of any one of claims 48-52, wherein a portion of the infant's body is elevated by a wedge.

54. The method of claim 53, wherein the wedge is the wedge of any one of claims 29-32.

55. The method of any one of claims 48-54, wherein the ears of the infant are covered by a head gear assembly configured to reduce noise exposure.

56. The method of claim 55, wherein the head gear assembly is the head gear assembly of any one of claims 1-10.

57. A method of reducing shock, vibration, or noise exposure to an infant during transportation, the method comprising:
(a) providing the mattress system of any one claims 27-43;
(b) providing the head gear assembly of any one of claims 1-10; and
(c) securing the head gear assembly to the infant and securing the infant to the mattress system.

58. The method of claim 57, wherein the mattress system is housed within an incubator chamber.

59. The method of claim 57 or 58, wherein specific portions of the infant's body are elevated using a plurality of support rolls.

60. The method of claim 59, wherein the support rolls are the support rolls of any one of claims 11-17.

61. A method of reducing shock or vibration while providing elevation to specific portions of an infant during transportation, the method comprising:
(a) providing the mattress system of any one claims 25-39;
(b) providing a plurality of support rolls of any one of claims 11-17; and
(c) securing the infant to the mattress system using the plurality of straps of the mattress system.

62. The method of claim 61, wherein the mattress system is housed within an incubator chamber.

63. The method of claim 61 or 62, wherein the ears of the infant are covered by a head gear configured to reduce noise.

64. The method of claim 57, wherein the head gear assembly is the head gear assembly of any one of claims 1-10.

65. A method of reducing the risk of intraventricular hemorrhage and/or persistent pulmonary hypertension to an infant during transportation, the method comprising:
(a) providing the mattress system of any one claims 27-43;
(b) providing the head gear assembly of any one of claims 1-10;
(c) providing a plurality of support rolls of any one of claims 11-17; and
(d) securing the infant to the mattress system using the plurality of straps of the mattress system.
